(19) United States
(12) Patent Application Publication
(10) Pub. No.: US 2017/0202579 A1
(43) Pub. Date: Jul. 20, 2017

(54) ANATOMICAL SPECIMEN COLLECTION DEVICE AND SYSTEM

(71) Applicants: John M. ABRAHAMS, Scarsdale, NY (US); Michael R. BIELSKI, Manorville, NY (US); Don HANNULA, San Luis Obispo, CA (US); Osteomedix, Inc., Manorville, NY (US)

(72) Inventors: John M. Abrahams, Scarsdale, NY (US); Michael R. Bielski, Manorville, NY (US); Don Hannula, San Luis Obispo, CA (US)

(73) Assignee: Osteomedix, Inc., Manorville, NY (US)

(21) Appl. No.: 15/328,671

(22) PCT Filed: Jul. 24, 2015
(86) PCT No.: PCT/US2015/042017

§ 371 Date: Jan. 24, 2017

Related U.S. Application Data
(60) Provisional application No. 62/030,882, filed on Jul. 30, 2014.
(60) Provisional application No. 62/028,552, filed on Jul. 24, 2014.

(51) Int. Cl. A61B 17/56; A61M 1/00

(52) U.S. Cl. CPC A61B 17/56; A61M 1/0056; A61M 1/0035; A61B 2217/007; A61B 2217/005

(57) ABSTRACT

A bone dust collection device includes a bone dust collection structure defining a longitudinal axis and having a proximal end and a distal end. The bone dust collection structure has a transverse plate member connected at or in the vicinity of the proximal end to a cylindrical wall defining an aperture at the distal end for receiving and collecting bone dust. The aperture extends proximally and includes a filtration member disposed therein. The filtration member causes at least partial separation of fluid from the bone dust via the fluid passing proximally through the filtration member. The bone dust collection structure enables removal of bone dust collected therein. A method of collecting an anatomical specimen includes generating an anatomical specimen in particulate form at a surgical site of a subject; collecting the particulate accumulated in the collection device; and packing the collected particulate into a region of interest in the subject.

### CROSS-REFERENCE TO RELATED APPLICATIONS

This application claims priority to, and benefit of, U.S. Provisional Patent Application No. 62/028,552 filed on Jul. 24, 2015, entitled “ANATOMICAL SPECIMEN COLLECTION DEVICE AND SYSTEM”, by John M. Abrahams and to U.S. Provisional Patent Application No. 62/030,882 filed on Jul. 30, 2015 of the same title, by John M. Abrahams et al., the entire contents of both of which are incorporated herein by reference.

### BACKGROUND

1. Technical Field

The present disclosure relates to anatomical specimen collectors.

2. Discussion of Related Art

During orthopaedic surgery, bone is drilled and bone dust is formed. During the surgical procedure, bone replacement is performed by harvesting bone dust from a subject other than the patient or subject in the form of allograft. In addition to the expense of supplying bone replacement from a source other than the patient or subject, the equipment used for such procedures is bulky and difficult to work with.

### SUMMARY

The present disclosure relates to novel and non-obvious device, system and method for harvesting an anatomical specimen such as bone dust from a subject and replacing the anatomical specimen in the subject.

More particularly, the present disclosure relates to a bone dust collection device that includes a bone dust collection structure defining a longitudinal axis and having a proximal end and a distal end. The bone dust collection structure has a transverse plate member connected at or in the vicinity of the proximal end to a cylindrical wall defining an aperture at the distal end for receiving and collecting bone dust, the aperture extending proximally and including a filtration member disposed therein. The filtration member is configured and disposed wherein the filtration member causes at least partial separation of fluid from the bone dust via the fluid passing proximally through the filtration member. The bone dust collection structure is configured to enable removal of bone dust collected therein.

In one embodiment, at least one aperture may be defined in the transverse plate member that enables the fluid at least partially separated from the bone dust by the filtration member to pass therethrough. A flow interruption member may be configured and disposed with respect to the bone dust collection structure wherein flow of fluid through the at least one aperture of the bone dust collection structure occurs by suction pressure induced on the flow interruption member causing the flow interruption member to change position to enable flow through the at least one aperture defined in the transverse plate member.

In one embodiment, least another aperture may be defined in the transverse plate member wherein the flow interruption member includes a shaft member disposed through the at least another aperture wherein the change of position of the flow interruption member occurs via the suction pressure induced on the flow interruption member to enable flow through the at least one aperture defined in the transverse plate member.

In one embodiment, the bone dust collection device may further include a syringe including a plunger in fluid communication with the bone dust collection structure wherein the suction pressure is caused by retraction of the plunger away from the bone dust collection structure to enable flow through the at least one aperture defined in the transverse plate member.

In one embodiment, advancement of the plunger may cause the flow interruption member to interrupt flow from the bone dust collection structure. The bone dust collection device may further include a conduit in fluid communication with the bone dust collection structure, wherein advancement of the plunger causes the fluid received through the at least one aperture defined in the transverse plate member to discharge through the conduit away from the bone dust collection device.

In one embodiment, the conduit may be structured such that the conduit discharges the fluid in a direction generally perpendicular to the direction of fluid flow through the filtration member.

In still a further embodiment, the bone dust collection device may further include another flow interruption device configured and disposed with respect to the flow interruption device and with respect to the bone dust collection structure wherein suction pressure induced on the another flow interruption device causes the another flow interruption device to be in a closed position and causes the flow interruption member to change position to enable flow through the at least one aperture defined in the transverse plate member.

In a further embodiment, the bone dust collection device may further include another flow interruption device configured and disposed with respect to the flow interruption device and with respect to the bone dust collection structure wherein the another flow interruption device is a plug member to seal off flow therethrough during connection of the bone dust collection device to a continuous vacuum system. and suction pressure induced on the flow interruption device causes the flow interruption member to change position to enable flow through the at least one aperture defined in the transverse plate member.

In yet another embodiment, the bone dust collection device may further include a bone dust removal structure defining an aperture having a proximal end and a distal end. The aperture may be configured and disposed for facilitating transport of bone dust from the distal end of the bone dust removal structure to the bone dust collection structure.

In a still further embodiment, the bone dust removal structure may include a nozzle that defines the aperture of the bone dust removal structure, the nozzle defining a proximal end and a distal end, wherein the nozzle facilitates transport of the bone dust to the bone dust collection structure. In yet another embodiment, the nozzle may be removably attached to the bone dust collection structure, wherein removal of the nozzle from the bone dust collection structure enables removal of bone dust collected within the bone dust collection structure.

In one embodiment, the bone dust collection device may further include a guide member configured and disposed with respect to the suction source and the bone dust collection structure for enabling a user to manually control the location of the bone dust removal structure during a surgical procedure.

In one embodiment, the flow interruption member may be an umbrella valve. In yet another embodiment, the another flow interruption member is a check valve.

The present disclosure relates also to a bone dust collection device that includes a bone dust collection structure defining a proximal end and a distal end, the bone dust collection structure including a filtration member at the proximal end and defining an aperture at the distal end for receiving and collecting bone dust, the filtration member causing at least partial separation of fluid from the bone dust via the fluid passing proximally through the filtration member; and a bone dust removal structure defining an aperture having a proximal end and a distal end, the aperture configured and disposed for facilitating transport of bone dust from the distal end of the bone dust removal structure to the bone dust collection structure, the bone dust removal structure removably attached to the bone dust collection structure, wherein removal of the bone dust removal structure from the bone dust collection structure enables removal of bone dust collected within the bone dust collection structure.

The present disclosure relates also to an anatomical collection system that includes an anatomical specimen collection structure defining a proximal end and a distal end, the anatomical specimen collection structure including a filtration member at the proximal end and defining an aperture at the distal end for receiving and collecting an anatomical specimen, the filtration member causing at least partial separation of fluid from the anatomical specimen via the fluid passing proximally through the filtration member; and an anatomical specimen removal structure defining an aperture having a proximal end and a distal end, the aperture configured and disposed for facilitating transport of the anatomical specimen from the distal end of the anatomical specimen removal structure to the anatomical specimen collection structure. The anatomical specimen removal structure is removably attached to the anatomical specimen collection structure wherein detachment of the anatomical specimen removal structure from the anatomical specimen collection structure enables removal of the anatomical specimen collected within the anatomical specimen collection structure.

In one embodiment, the anatomical specimen is bone dust.

The present disclosure relates also to a method of collecting an anatomical specimen that includes generating an anatomical specimen in particulate form at a surgical site of a subject; irrigating the surgical site to suspend the particulate in a particulate fluid mixture; disposing a tip of a collection device in the particulate fluid mixture; aspirating the collection device to filter and separate the particulate from the particulate fluid mixture therein accumulating the particulate in the collection device; and collecting the particulate accumulated in the collection device.

The method may further include packing the collected particulate into a region of interest in the subject.

In one embodiment, the collecting the particulate accumulated in the collection device is preceded by disassembling the collection device, and the method may further include reassembling the collection device; generating additional anatomical specimen in particulate form at a surgical site of the subject; irrigating the surgical site to suspend the particulate in a particulate fluid mixture; disposing the tip of the collection device in the particulate fluid mixture; aspirating the collection device to filter and separate the particulate from the particulate fluid mixture therein accumulating the particulate in the collection device; and collecting the particulate accumulated in the collection device.

The method may further include packing the additionally collected particulate into a region of interest in the subject.

In one embodiment, the aspirating of the collection device may include connecting the collection device to a vacuum source, which in one embodiment, may be in an operating room, wherein the vacuum source is in fluid communication with the fluid in the particulate fluid mixture to filter and separate the particulate from the particulate fluid mixture.

In one embodiment, the aspirating of the collection device may include actuating a syringe in fluid communication with the collection device and in fluid communication with the particulate fluid mixture wherein the actuating of the syringe effects filtering and separating the particulate from the particulate fluid mixture.

### BRIEF DESCRIPTION OF THE DRAWINGS

The advantages of the embodiments of the present disclosure will become apparent from the following detailed description of the various embodiments of the present disclosure with reference to the drawings wherein:

FIG. 1 is a transparent perspective view of a bone dust collection system according to one embodiment of the present disclosure;

FIG. 2 is an exploded view of a bone dust collection device that is included within the bone dust collection system of FIG. 1;

FIG. 3 is a profile view of one embodiment of a bone dust collection device that may be included within the bone dust collection system of FIG. 1;

FIG. 4 is a partial profile cross-sectional view of the bone dust collection system of FIGS. 1 and 2 having one embodiment of a filtration member according to the present disclosure;

FIG. 5 is a cross-sectional enlarged view of the bone dust collection housing and valve support structure of the bone dust collection device of FIG. 3 having an embodiment of a filtration member according to the present disclosure;

FIG. 6A is an end view of a filter that is included within the bone dust collection device of FIGS. 1-5;

FIG. 6B is a side view of the filter of FIG. 6A;

FIG. 6C is a view of Detail 6C of FIG. 6B;

FIG. 6D is a proximal perspective view of the filter of FIGS. 6A-6C;

FIG. 7A is an end view of one embodiment of a nozzle that may be included within the bone dust collection device of the bone dust collection system of FIGS. 1-5;

FIG. 7B is a cross-sectional view of the nozzle of FIG. 7A taken along cross-section line 7B-7B;

FIG. 8A is an end view of another embodiment of a nozzle that may be included within the bone dust collection device of the bone dust collection system of FIGS. 1-5;

FIG. 8B is a cross-sectional view of the nozzle of FIG. 8A taken along cross-section line 8B-8B;

FIG. 9A is an end view of one embodiment of a valve cap that that may be included with the bone dust collection device of the bone dust collection system of FIGS. 1-5;

FIG. 9B is a cross-sectional view of the valve cap of FIG. 9A taken along section line 9B-9B;

FIG. 10 is an exploded partially transparent view of a handle assembly that may be included as part of the bone dust collection device of the bone dust collection system of FIGS. 1-5;

FIG. 10A is a cross-sectional view of the handle assembly of FIG. 10;

FIG. 11 is a perspective, partially transparent view of the handle assembly of FIGS. 10 and 10A and the bone dust collection device of FIG. 3 that includes a plug seal;

FIG. 12 is a perspective, partially transparent view of the handle assembly of FIG. 11 and the bone dust collection device of FIGS. 1-2 and 4-5 that includes a check valve seal;

FIG. 13 is a partially transparent view of one embodiment of a self-contained bone dust collection device according to the present disclosure that includes a check valve and a flow diversion member for diverting fluid flow from the bone dust collection device;

FIG. 13A is a partially transparent view of another embodiment of the self-contained bone collection device of FIG. 13;

FIG. 14 is a zoomed out partial profile cross-sectional view of the bone dust collection system of FIG. 1-3;

FIG. 15A is a detailed view of an extraction flow interruption member illustrated in the form of an umbrella valve that is positioned on an upper surface of the housing in a closed position;

FIG. 15B is a detailed view of the umbrella valve positioned on the upper surface of the housing in an open position to enable flow of fluid past the umbrella valve;

FIG. 16 is a cross-sectional view of the bone dust collection device when the umbrella valve is in the closed position as illustrated in FIG. 15A;

FIG. 17 is a cross-sectional view of the bone dust collection device when the umbrella valve is in the open position for permitting passage of fluid past the umbrella valve;

FIG. 18 is a cross-sectional view of the bone dust collection device when the umbrella valve is in the closed position for discharging and diverting fluid which has been collected by syringe;

FIG. 19 is a cross-sectional view of another embodiment of the bone dust collection device having the alternate filtration member shown in FIG. 4 and wherein the umbrella valve is in the open position;

FIG. 20 is cross-sectional view of the bone dust collection device of FIG. 19 wherein the umbrella valve is in the closed position and fluid is diverted through a check valve;

FIG. 21 is a cross-sectional view of the bone dust collection device having the filtration member of FIG. 5 in an inverted position to allow removal of collected bone dust;

FIG. 22 is a cross-sectional view of the bone dust collection device of FIG. 19 in an inverted position to allow removal of collected bone dust;

FIG. 23 is a perspective transparent view of a kit package that houses bone dust collection devices according to embodiments of the present disclosure;

FIG. 24 is a perspective view of the bone dust collection device of FIG. 3 having a plug member to seal off flow during connection of the bone dust collection device to a continuous vacuum system;

FIG. 25 is a perspective view of the bone dust collection device of FIGS. 1-2 and 4-5 having a fluid flow diversion member and a check valve wherein fluid flow is directed vertically downward;

FIG. 26 is a cross-sectional view of the bone dust collection device of FIG. 25;

FIG. 27 is a perspective view of the bone dust collection device of FIGS. 25 and 26 wherein the fluid flow is directed horizontally;

FIG. 28 is a perspective view of the bone dust collection device of FIG. 24 wherein the plug member is replaced by a flow diversion member to enable diversion of fluid back to a surgical site;

FIG. 29 is a transparent elevation view of the bone dust collection device of FIG. 28;

FIG. 30 is a perspective view of the bone dust collection device of FIG. 28 wherein a tubular portion of the flow diversion member has been removed;

FIG. 31 is a perspective view of the bone dust collection device of FIG. 30 wherein the flow diversion member is oriented at an angle to enable diversion of fluid in an alternative direction with respect to a surgical site; and

FIG. 32 is a perspective view of the bone dust collection device of FIG. 30 wherein the flow diversion member is oriented at an angle to enable diversion of fluid in yet another alternative direction with respect to a surgical site.

### DETAILED DESCRIPTION

To advance the state of the art of bone dust collection, the present disclosure relates to a bone dust collection system where a user, e.g., a surgeon or other medical professional, is able to simply collect bone dust using a hand-held device during aspiration from a surgical site.

As defined herein, a bone dust collection device refers to an assembly of components which may either be employed independently or in a self-contained manner to collect bone dust while a bone dust collection system refers to utilization of the device and its assembly of components in conjunction with an external suction source such as via fluid communication with an operating room suction or vacuum system. The suction or vacuum system may also be located outside of an operating room under appropriately certified conditions. The device can be used to collect other anatomical specimen besides bone dust, such as, for example, bone marrow or other biological cells.

The bone dust collection device enables collection of bone dust, defined herein as generally in the range of about 25 micron to about 600 micron cross-section bone particles, during a surgical procedure in order to harvest and use the bone dust for bone fusion mass in orthopedic surgery of the same patient. In actuality, the bone dust collection device enables collection of bone dust of sizes generally known to be created during surgical procedures.

The device generally may be applied to one or more medical procedures performed on a single patient. The device may be utilized as a stand-alone bone collection device that includes a syringe or the device may be adapted as a bone collection system by fluidically coupling the device to a vacuum source such as commonly found in an operating room environment. The device may be used multiple times on the same patient during the same operating procedure but is discarded after completion of usage.

The output of the device may be a “plug” of bone that can be held with forceps and placed into an intervertebral cage or used for any other procedure that requires bone or fusion construct. The output of the device may also be bone dust that is concentrated and can be manually placed into an intervertebral cage or construct or placed therein using instruments.

The device is configured to allow fluids, e.g., blood and saline, to pass through a separator or filtration member, e.g., a filter or a valve mechanism, thus separating bone dust from fluids. The bone dust collection device aspirates surgical fluid and filters out bone dust for use during a surgical procedure.

The bone dust so collected may be processed by the user during the procedure wherein the processing of the bone dust may include cleaning, compacting, and transfer of the bone dust.

As described above, the bone dust collection device may be disposed of, and in most cases is intended to be discarded, following usage.

After using a surgical drill, the user aspirates surgical fluid to filter and then harvest bone dust; Fluid is discarded and bone dust is removed from a collection chamber defined within the bone dust collection device. Bone dust may be used for fusion or reconstruction procedures. Procedures include, but are not limited to, e.g., spinal fusion, long bone fusion, cranial reconstruction, craniofacial reconstruction, orthopaedic reconstruction, or dental reconstruction.

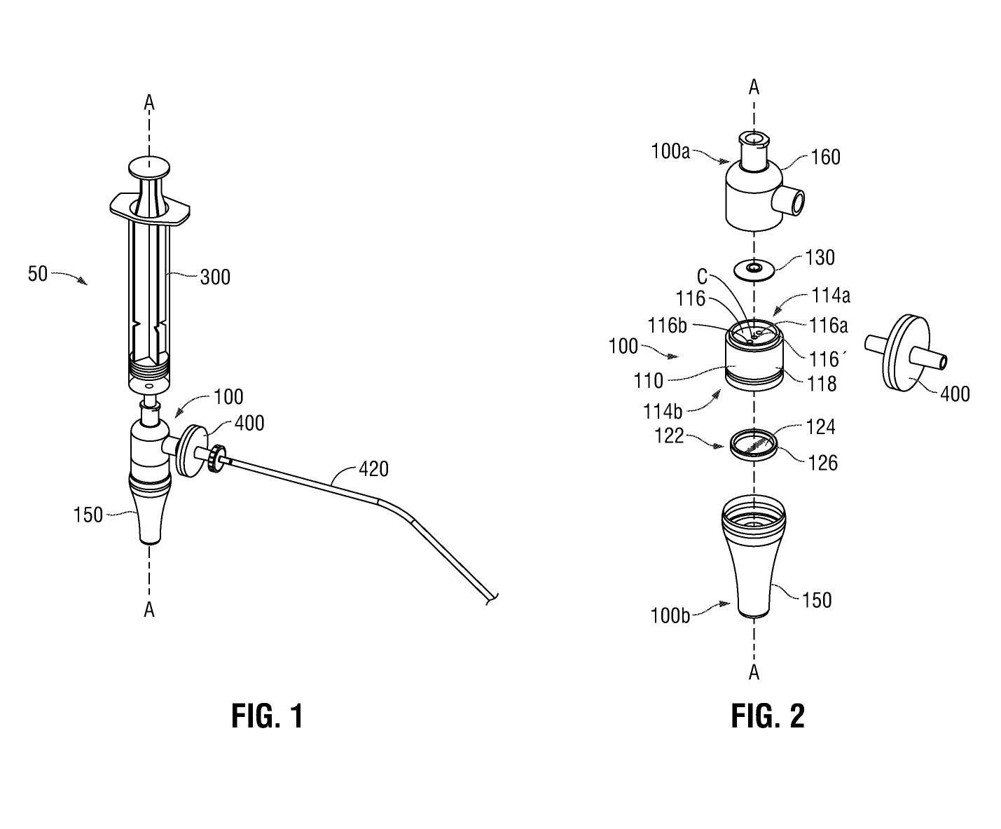

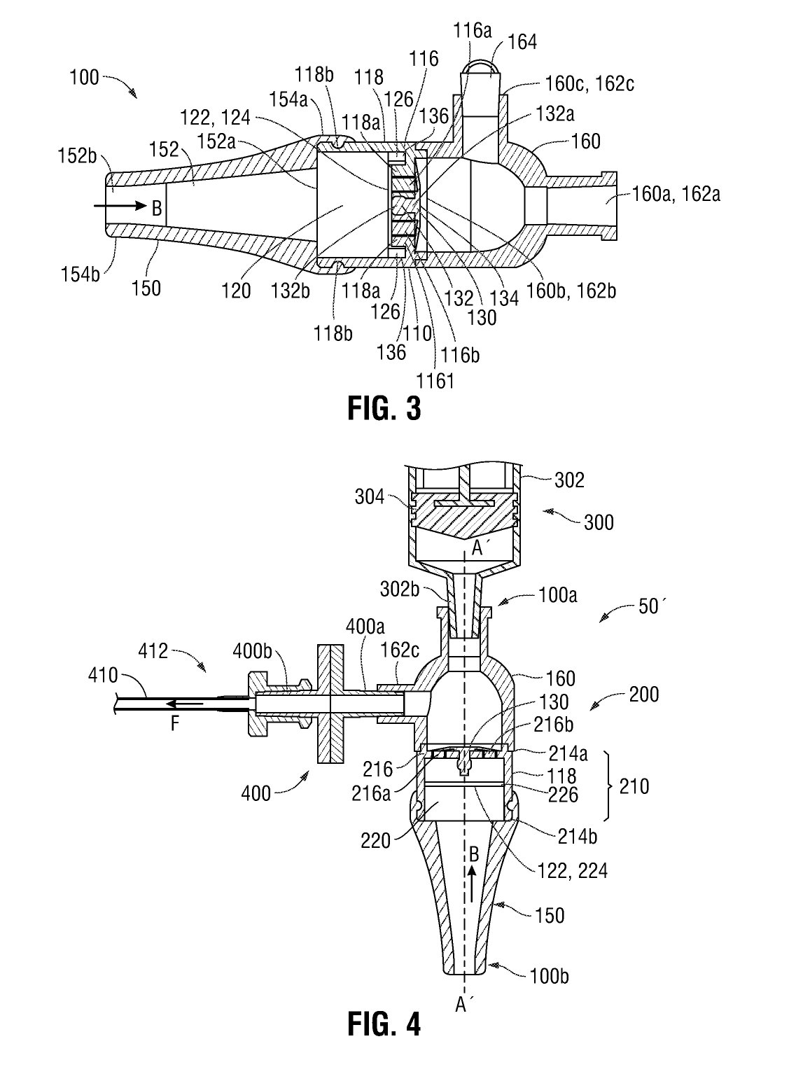

Turning now to FIGS. 1, 2 and 3, there is illustrated a bone dust collection system 50 that includes a bone dust collection device according to one embodiment of the present disclosure. More particularly, bone dust collection device 100 includes a cylindrically-shaped bone dust collection structure 110 that extends from a proximal end 114a to a distal end 114b of the bone dust collection structure 110. The proximal end 114a of the bone dust collection structure 110 is configured as a valve support plate 116 such that the bone dust collection structure 110 also serves as a valve support structure. Cylindrical wall 118 extends distally from the valve support plate 116 to define a central aperture and volume 120 for receiving and collecting bone dust (see FIG. 3).

As best illustrated in FIG. 2 and FIG. 3, the valve support structure or bone dust collection structure 110 includes a valve supporting surface or plate 116 near the proximal end 114a.

The central aperture 120 extends proximally towards proximal end 114a of the bone dust collection structure 110 and includes a filtration member 122 disposed therein that may be part of a filter assembly 124. The filter assembly 124 may include a ring-shaped filter mounting member 126 that is configured to be disposed within the central aperture 120.

As explained in more detail below, the filtration member 122 and the filter assembly 124 are configured and disposed wherein the filtration member 122 causes at least partial separation of fluid from the bone dust via the fluid passing proximally through the filtration member 122. Thus, the bone dust collection structure 110 is configured to enable removal of bone dust collected therein.

As best shown in FIG. 2, the bone dust collection device 100 defines a central longitudinal axis A-A that extends from a proximal end 100a to a distal end 100b. At least one fluid flow aperture 116a is defined in the transverse plate member 116 that enables the fluid at least partially separated from the bone dust by the filtration member 122 to pass therethrough. A fluid extraction valve shaft aperture 116′ is also defined in the transverse plate member 116 generally through the center C of the transverse plate member 116 through which longitudinal axis A-A passes. A second fluid flow aperture 116b may also be defined in the transverse plate member 116 on a side of the longitudinal axis A-A opposite to the first fluid flow, e.g., at a position 180° from the first fluid flow aperture 116a, which is at position 0°. Additional fluid flow apertures (not shown) may also be positioned at 90° or 270° or other suitable locations if desired. It should be noted also that the bone dust collection structure 110 may be oriented such that the first and second fluid flow apertures 116a and 116b may coincide with the 90° and 270° positions, respectively, or vice versa. Such interchangeability of the orientation of the first and second fluid flow apertures 116a and 116b is taken advantage of throughout the description which follows where necessary or desirable to explain the operation of the bone dust collection device 100.

The bone dust collection device 100 further includes a fluid extraction flow interruption member 130 that is configured and disposed with respect to the bone dust collection structure 110 wherein, as explained in more detail below, flow of fluid through at least one aperture or, for example, fluid flow apertures 116a and 116b, of the bone dust collection structure 110 occurs by suction pressure induced on the fluid extraction flow interruption member 130 causing the flow interruption member 130 to change position to enable flow through fluid flow apertures 116a and 116b.

As indicated above, fluid extraction valve shaft aperture 116′ is defined in the transverse plate member 116. As shown in FIG. 3, the extraction flow interruption member 130 may be an umbrella valve as shown and which includes a shaft member 132 that is disposed through the fluid extraction valve shaft aperture 116′. The extraction flow interruption member 130 includes a diaphragm 134 that is formed with the shaft member 132. The diaphragm 134 is made from an elastomeric and flexible material and which overlays the fluid flow apertures 116a and 116b on the proximal side of transverse plate member 116 such that the change of position of the extraction flow interruption member 130 occurs via the suction pressure induced on the flow interruption member, and more particularly on the diaphragm 134, such that the diaphragm 134 flips away from the transverse plate member 116 to expose the fluid flow apertures 116a and 116b, thereby enabling flow through the apertures 116a and 116b. The shaft member 132 includes a proximal portion 132a and a distal portion 132b wherein the distal portion 132b defines a diameter that is greater than the diameter of the proximal portion 132a. The fluid extraction valve shaft aperture 116′ is thus shaped accordingly to define a proximal narrow diameter portion and a distal wide diameter portion to match the proximal and distal portions 132a and 132b of the shaft member 132.

In the embodiment of the housing 110 illustrated in FIG. 3, a portion 1161 of the transverse plate member 116 extends distally into the central aperture 120 to define a circumferential channel 136 between inner surface 118a of the housing wall 118 and the distally extending portion 1161 of the transverse plate member 116. The filter assembly 124 is configured and disposed wherein the ring-shaped filter mounting member 126 is received in the circumferential channel 136 such that the filtration member 122 overlays the distal ends of the fluid flow apertures 116a and 116b.

The bone dust collection device 100 may further include a bone dust removal structure 150 that defines an aperture 152 having a proximal end 152a and a distal end 152b. In one embodiment, the bone dust removal structure 150 may include, or be formed in the shape of, a nozzle wherein proximal end 154a of the nozzle 150 may engage with outer surface 118b of housing wall 118 for structural stability of the nozzle 150 during usage for collecting bone dust. The aperture 152 of the nozzle 150 is configured and disposed for facilitating transport of bone dust, or a mixture of bone dust and irrigating fluid such as saline solution, as represented by arrow B, from the distal end 152b of the bone dust removal structure or nozzle 150 to the central aperture 120 of the bone dust collection structure 110. When in the form of a nozzle, the nozzle thus defines the aperture 152 of the bone dust removal structure 150. The nozzle defines the proximal end 152a and distal end 152b, wherein the nozzle facilitates transport of the bone dust B, which includes fluid from the surgical site, to the bone dust collection structure 110. The nozzle may be removably attached to the bone dust collection structure 110 by disengaging from outer surface 118b of housing wall 118. Removal of the nozzle 150 from the bone dust collection structure 110 enables removal of bone dust collected within the bone dust collection structure 110, as described in more detail below. The nozzle 150 may be made from flexible, elastomeric materials, such as, but not limited to, silicone or urethane.

The bone dust collection device 100 may further include a tee-shaped port member or cap 160 that defines a bone dust collection interface aperture 162b at a distal portion 160b of the tee-shaped port member or cap 160. The tee-shaped port member or cap 160 may further define a suction device interface aperture 162a at a proximal portion 160a. Additionally, tee-shaped port member or cap 160 defines a fluid diversion aperture 162c at branch connection 160c. As described in more detail below, when in operation, the bone dust collection device 100 in FIG. 3 is connected to a continuous suction source, such as an operating room suction system, at suction device interface aperture 162a. When in this mode of operation for collecting bone dust, the fluid diversion aperture 162c is sealed via a plug 164 inserted therethrough. Fluid from the surgical site is separated from the bone dust (arrow B) by the filtration member 122 and passes through to the suction device interface aperture 162a. Suction is maintained by the presence of the plug 164.

FIG. 4 is a cross-sectional view of the bone dust collection system of FIGS. 1 and 2 having another embodiment of a bone dust collection device according to the present disclosure. More particularly, bone dust collection system 50′ is identical to bone dust collection system 50 except that bone dust collection structure 110 is replaced by bone dust collection structure 210 which differs from bone dust collection structure 110. For simplicity, only those features of bone dust collection structure 210 which differ from the corresponding features of bone dust collection structure 110 are described. Additionally, those features are numbered as 210 instead of 110, 216 instead of 116, etc. Reference to FIG. 3 is implied where features not explicitly shown in FIG. 4 are described.

Bone dust collection device 200 includes cylindrically-shaped bone dust collection structure 210 that extends from proximal end 214a to distal end 214b of the bone dust collection structure 210. The proximal end 214a of the bone dust collection structure 210 is also configured as a valve support plate 216 such that the bone dust collection structure 210 also serves as a valve support structure in the same manner as described above with respect to bone dust collection device 100. Cylindrical wall 118 extends distally from the valve support plate 216 to define a central aperture and volume 220 for receiving and collecting bone dust.

Valve support structure or bone dust collection structure 210 includes valve supporting surface or plate 216 near the proximal end 214a.

The central aperture 220 extends proximally towards proximal end 214a of the bone dust collection structure 210 and includes filtration member 122 disposed therein that may be part of filter assembly 224. The filter assembly 224 may now include ring-shaped filter mounting member 226 that is configured to be disposed within the central aperture 220.

Again, as explained in more detail below, the filtration member 122 and the filter assembly 224 are configured and disposed wherein the filtration member 122 causes at least partial separation of fluid from the bone dust via the fluid passing proximally through the filtration member 122. The filter assembly 224 and filtration member 122 are mounted within the central aperture 220 wherein the ring-shaped filter mounting member 226 has an outside diameter approximately corresponding to the inside diameter of the cylindrical wall 118 such that the ring-shaped filter mounting member 226 may be disposed in a snug fit within the bone dust collection structure 210. Thus, the bone dust collection structure 210 is also configured to enable removal of bone dust collected therein.

Although illustrated as an assembly of filtration member 122 and the filter mounting member 226, the filter assembly 224 may be a molded construction. In either case, the filtration member 122 may be a mesh with an over-under weave knit material or be a molded membrane with small holes or apertures formed therein, on the order of 100 microns (μm) in cross-sectional distance. The filtration member 122 may be made from, but not limited to, materials such as fiberglass, copper or stainless steel. The ring-shaped filter mounting member 226 may be made from a thermoplastic elastomer such as polyvinyl chloride and urethane.

Bone dust collection device 200 also defines a central longitudinal axis A′-A′ that extends from proximal end 100a to distal end 100b. At least one fluid flow aperture 216a is defined in transverse plate member 216 that enables the fluid at least partially separated from the bone dust by the filtration member 122 to pass therethrough. Fluid extraction valve shaft aperture 216′ is also defined in the transverse plate member 216 generally through the center C of the transverse plate member 216 through which longitudinal axis A′-A′ passes. Second fluid flow aperture 216b may also be defined in the transverse plate member 216 again on a side of the longitudinal axis A′-A′ opposite to the first fluid flow, e.g., at a position 180° from the first fluid flow aperture 216a, which is at position 0°. Additional fluid flow apertures (not shown) may also be positioned at 90° or 270° or other suitable locations if desired. It should be noted again that the bone dust collection structure 210 may also be oriented such that the first and second fluid flow apertures 216a and 216b may coincide with the 90° and 270° positions, respectively, or vice versa. Such interchangeability of the orientation of the first and second fluid flow apertures 216a and 216b is taken advantage of throughout the description which follows where necessary or desirable to explain the operation of the bone dust collection device 200.

The bone dust collection device 200 further includes also fluid extraction flow interruption member 130 that is configured and disposed with respect to the bone dust collection structure 210 wherein, as explained in more detail below, flow of fluid through at least one aperture or, for example, fluid flow apertures 216a and 216b, of the bone dust collection structure 210 occurs by suction pressure induced on the fluid extraction flow interruption member 130 causing the flow interruption member 130 to change position to enable flow through fluid flow apertures 216a and 216b.

As indicated above, fluid extraction valve shaft aperture 216′ is defined in the transverse plate member 216. As shown in FIG. 3, the extraction flow interruption member 130 may be an umbrella valve as shown and which includes shaft member 132 that is disposed through the fluid extraction valve shaft aperture 216′. The extraction flow interruption member 130 again includes diaphragm 134 that is formed with the shaft member 132. The diaphragm 134 is again made from an elastomeric and flexible material and which overlays the fluid flow apertures 216a and 216b on the proximal side of transverse plate member 216. Again, the change of position of the extraction flow interruption member 130 occurs via the suction pressure induced on the flow interruption member, and more particularly on the diaphragm 134, such that the diaphragm 134 flips away from the transverse plate member 216 to expose the fluid flow apertures 216a and 216b, thereby enabling flow through the apertures 216a and 216b. The shaft member 132 includes again proximal portion 132a and distal portion 132b wherein the distal portion 132b defines a diameter that is greater than the diameter of the proximal portion 132a. However, in contrast to fluid extraction valve shaft aperture 116′, fluid extraction valve shaft aperture 216′ is thus shaped accordingly to define a narrow diameter portion to match the proximal portion 132a of the shaft member 132. The transverse plate member 216 is not characterized by the portion 1161 of the transverse plate member 116 that extends distally into the central aperture 120. Thus there is no circumferential channel 136 between inner surface 118a of the housing wall 118 and the distally extending portion 1161 of the transverse plate member 116.

The bone dust collection device 200 also includes the bone dust removal structure 150 mounted on cylindrical wall 118 and tee-shaped port member or cap 160 mounted to bone dust collection structure 210 at proximal end 214a in a similar manner as described above with respect to FIG. 3. However, a suction device 300 is now inserted into the suction device interface aperture 162a while a fluid flow diversion interruption device 400 is in fluid communication with fluid diversion aperture 162c at branch connection 160c.

Fluid flow diversion interruption device 400 is configured and disposed with respect to the flow interruption member 130 and with respect to the bone dust collection structure 210 wherein suction pressure induced on the flow interruption member 130 causes the flow interruption member 130 to change position to enable flow through the apertures 216a and 216b defined in the transverse plate member 216 while fluid flow diversion interruption device 400 is in a closed position.

In one embodiment, suction device 300 may be a syringe 302 including a plunger 304 in fluid communication with the bone dust collection structure 210 via distal port 302b of the syringe 302 inserted into the suction device interface aperture 162a of tee-shaped port member or cap 160. Suction pressure is caused by retraction of the plunger 304 in the proximal direction away from the bone dust collection structure 210 to enable flow through the apertures 216a and 216b defined in the transverse plate member 216.

Conversely, advancement of the plunger 304 distally causes the flow interruption member 130 to interrupt flow from the bone dust collection structure 210 while fluid is diverted through the fluid flow diversion interruption device 400 which includes an upstream port 400a that is inserted in fluid diversion aperture 162c of the tee-shaped port member or cap 160.

On downstream side 400b of fluid flow diversion interruption device 400, there is disposed a conduit 410 in fluid communication with the bone dust collection structure 210, wherein advancement of the plunger 304 causes the fluid (as represented by arrow F) received through the apertures 216a and 216b defined in the transverse plate member 216 to discharge through the fluid conduit 410 away from the bone dust collection device 200 in the direction of arrow F. Fluid communication between the fluid conduit 410 and the fluid flow diversion interruption device 400 may be established via a luer valve 412 connecting the fluid conduit 410 to the downstream side 400b. In one embodiment, the fluid flow diversion interruption device 400 may be a check valve installed so as to enable flow of fluid in the direction of arrow F and to prevent flow in the direction opposite to arrow F. In one embodiment, the conduit 410 is structured such that the conduit 410 discharges the fluid in a direction generally perpendicular, e.g., in the direction of arrow F, to the direction of fluid flow through the filtration member 122.

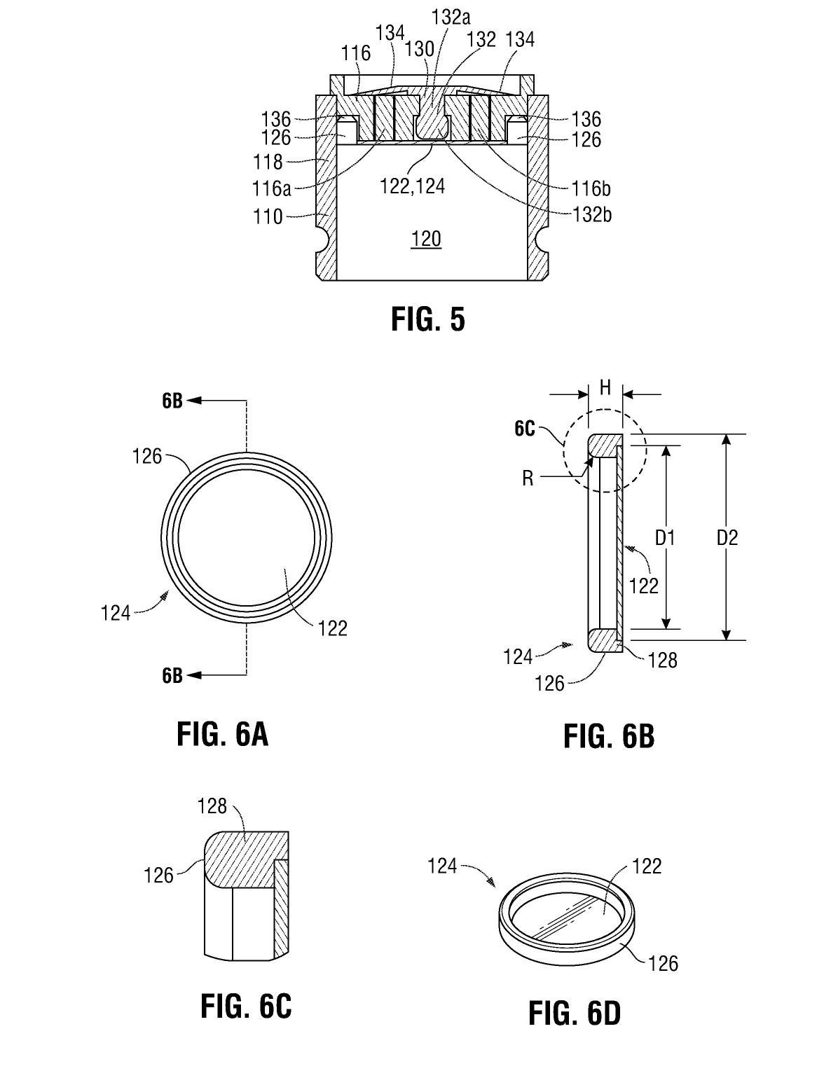

FIG. 5 is a cross-sectional enlarged view of the bone dust collection housing and valve support structure 110 of the bone dust collection device of FIG. 3 having an embodiment of the filtration member 122 and transverse plate member 116 with circumferential channel 136 described above with respect to FIG. 3.

FIG. 6A is an end view of the filter assembly 124 that is included within the bone dust collection device 200 of FIGS. 1-3 and 5. FIG. 6B is a side view of the filter assembly 124 of FIG. 6B illustrating wherein ring-shaped filter mounting member 126 includes a cylindrical wall 128 which is configured and disposed to be received within the circumferential channel 136 illustrated in FIGS. 3 and 5. The cylindrical wall 128 has a height dimension H above the filtration member 122. The cylindrical wall 128 may be characterized by a partially contoured profile at the proximal end having a radius of curvature R. The ring-shaped filter mounting member 126 may define an inner diameter D1 and an outer diameter D2.

FIG. 6C is a view of Detail 6C of FIG. 6B wherein ring-shaped filter mounting member 126 includes cylindrical wall 128.

FIG. 6D is a proximal perspective view of the filter assembly 124 of FIGS. 6A-6C.

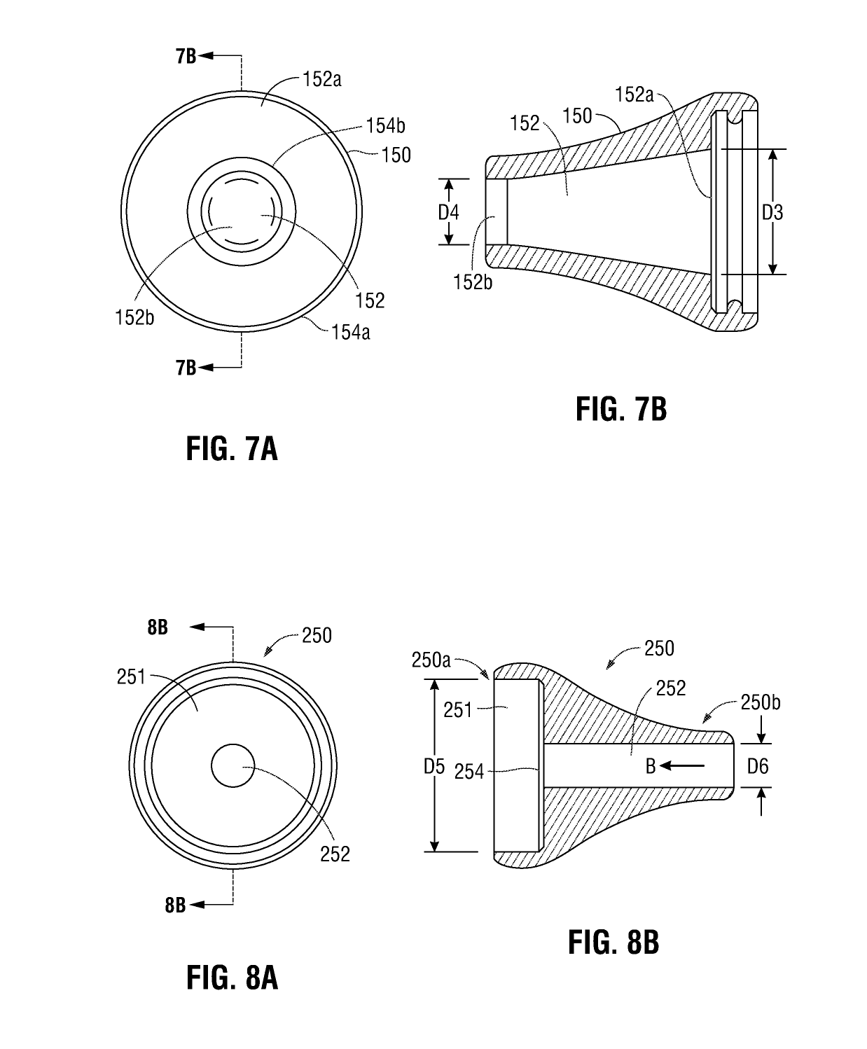

FIG. 7A is an end view of the bone dust removal structure 150 in the form of a nozzle that may be included within the bone dust collection device 100 or 200 of the bone dust collection systems 50 or 50′ of FIGS. 1-5.

FIG. 7B is a cross-sectional view of the nozzle 150 of FIG. 7A taken along cross-section line 7B-7B. The diameter D3 of central aperture 152 at proximal end 152a tapers in the distal direction to diameter D4 at distal end 152b such that diameter D3 is greater than diameter D4. Having the tapered configuration such that the diameter of the central aperture 152 increases from the distal end 152b to the proximal end 152a tends to minimize flow obstruction during operation of the bone collection device 100.

FIG. 8A is an end view of another embodiment of bone dust removal structure in the form of a nozzle 250 that may be included within the bone dust collection device 100 or 200 of the bone dust collection systems 50 or 50′ of FIGS. 1-5.

FIG. 8B is a cross-sectional view of the nozzle of FIG. 8A taken along cross-section line 8B-8B. More particularly, nozzle 250 defines a generally constant diameter D5 circular aperture 251 at proximal end 250a wherein the circular aperture 251 is configured and disposed to receive the filter assembly 124 (see FIG. 1-5). Nozzle 250 further defines another generally constant diameter D6 circular aperture 252 that extends distally from an interface 254 with circular aperture 251 wherein bone dust B is suctioned from distal end 250b. In a similar manner as with respect to nozzle 150, nozzle 250 may be made from flexible, elastomeric materials, such as, but not limited to, silicone.

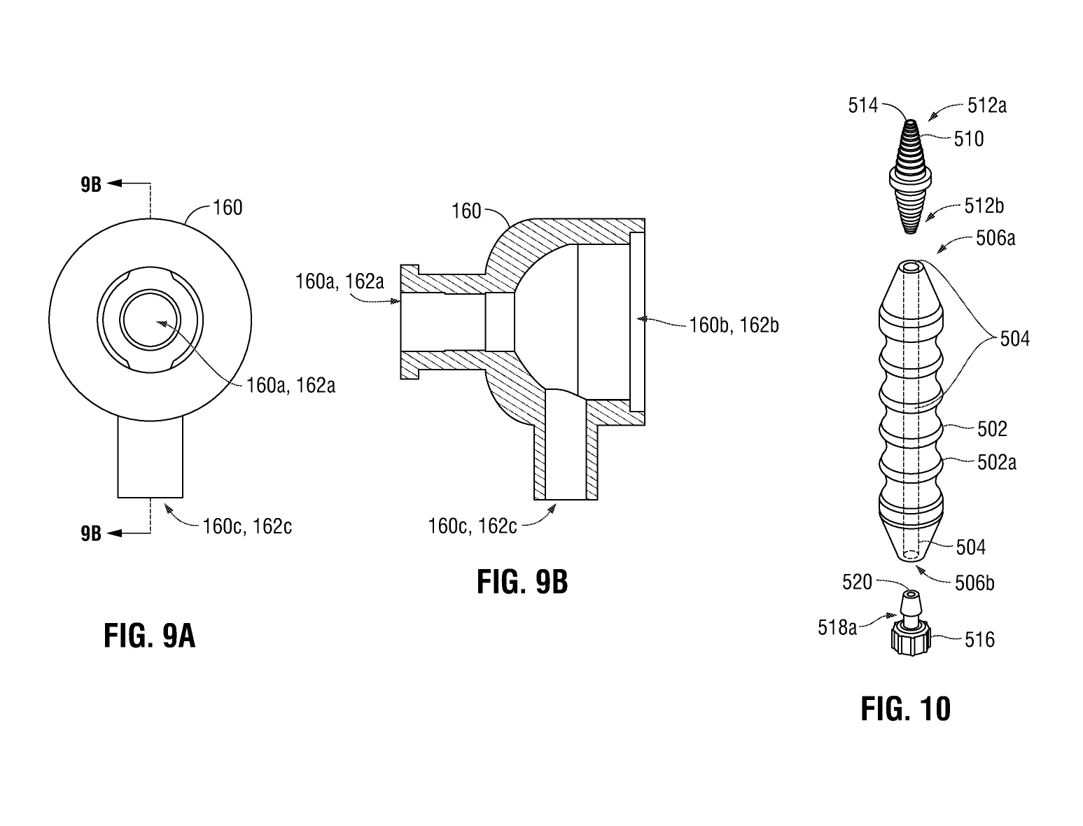

FIG. 9A is an end view of one embodiment of valve tee-shaped port member or cap 160 that that may be included with the bone dust collection device 100 or 200 of the bone dust collection systems 50 or 50′ of FIGS. 1-5.

FIG. 9B is a cross-sectional view of the valve cap tee-shaped port member or cap 160 of FIG. 9A taken along section line 9B-9B.

As described above with respect to FIG. 4, tee-shaped port member or cap 160 that defines bone dust collection interface aperture 162b at distal portion 160b of the tee-shaped port member or cap 160. The tee-shaped port member or cap 160 may further define suction device interface aperture 162a at proximal portion 160a. Additionally, tee-shaped port member or cap 160 defines fluid diversion aperture 162c at branch connection 160c at generally a right angle with respect to interface apertures 162a and 162b.

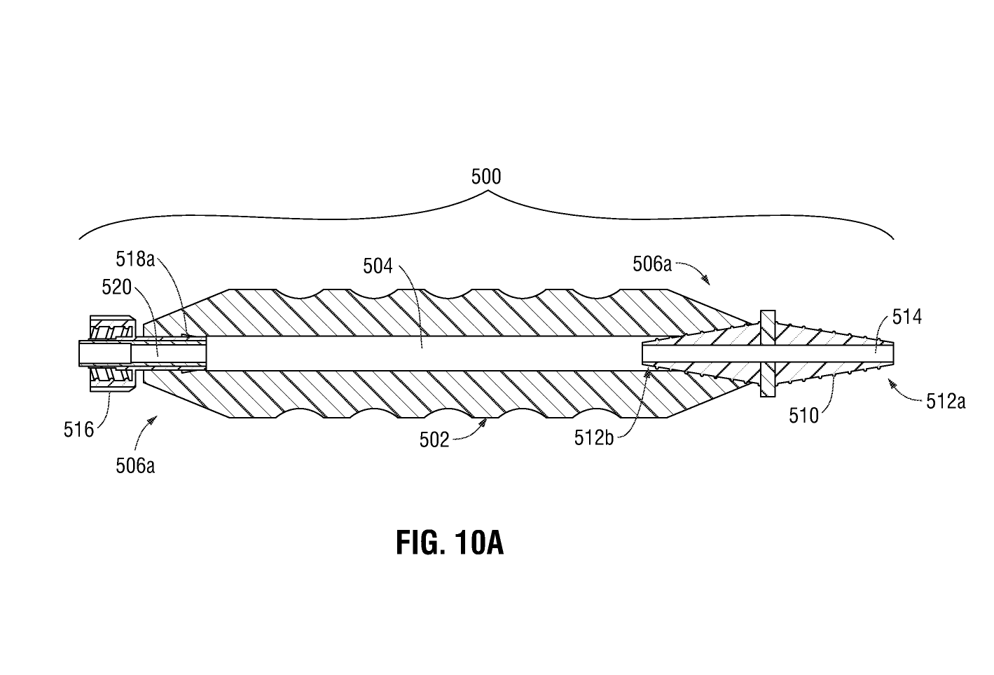

FIG. 10 is an exploded partially transparent view of a handle assembly 500 that may be included as part of the bone dust collection devices 100 or 200 of the bone dust collection systems 50 or 50′ of FIGS. 1-5. The handle assembly 500 includes a central handle member 502 having a generally cylindrical configuration with a knurled outer surface 502a having a series of ridges to assist a user in gripping the handle member 502. The central handle member 502 also defines a central bore or aperture 504 that extends from proximal end 506a of the handle member 502 to distal end 506b of the handle member 502.

The handle assembly 502 may further include a connector member 510 at proximal end 506a. The connector member 510 defines a barbed proximal end 512a and a barbed distal end 512b and a central bore 514 extending through each end. Distal barbed end 512b of connector member 510 is configured and disposed to engage with proximal end 506a of the central handle member 502 and be received within the central bore or aperture 504.

Additionally, the handle assembly 500 may include at distal end 506a of central handle member 502 a luer fitting 516 having a proximal barbed end 518a. The luer fitting 516 defines a central bore 520 such that the proximal barbed end 518a is configured to be received within the central bore or aperture 504.

FIG. 10A is a cross-sectional view of the handle assembly 500 of FIG. 10.

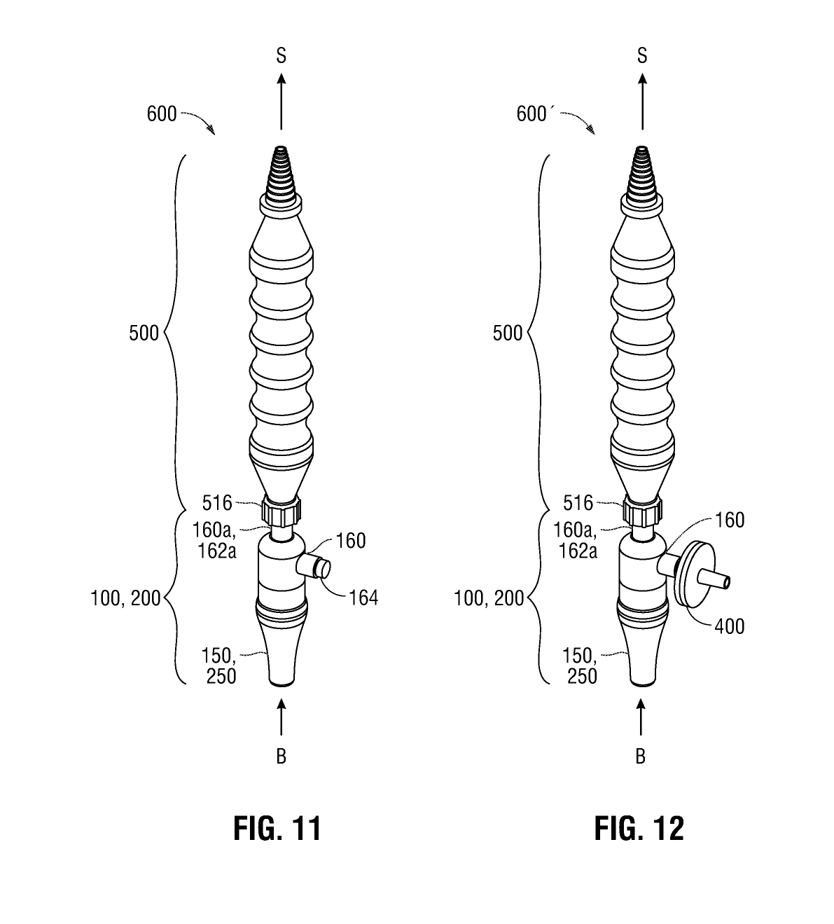

FIG. 11 is a perspective, partially transparent view of the handle assembly of FIGS. 10 and 10A and the bone dust collection device of FIG. 3 that includes a plug seal forming a bone dust collection system 600. As can be appreciated by the foregoing description of FIGS. 110 and 10A, when the handle assembly 500 is assembled as shown wherein the luer fitting 516, the central handle member 502 and the connector member 510 are connected in series, fluid communication is established from the luer fitting 516 to the connector member 510 via the central bore 520 of the luer fitting 516, the central bore 504 of the central handle member 502 and the central bore 514 of the connector member 510. In this configuration, suction is established through the handle assembly 500 in the proximal direction of arrow S by connection to a continuous suction source such as an operating room such system (not shown). Bone dust as represented by arrow B at bone dust removal structure 150 is collected by the bone dust collection device 100 or 200 by the establishment of the suction condition S. The fluid extraction flow interruption member 130 (see FIG. 3) is now in an open position to permit the passage of fluid through the handle assembly 500 while fluid flow through the fluid diversion aperture 162c at branch connection 160c is sealed via the plug member 164. Bone dust is collected in the bone dust collection device 100 or 200.

FIG. 12 is a transparent view of the handle assembly 500 of FIG. 11 and the bone dust collection device 100 or 200 of FIGS. 1-5 forming bone dust collection system 600′. As can be appreciated by the foregoing description of FIG. 11, when the handle assembly 500 is assembled as shown wherein the luer fitting 516, the central handle member 502 and the connector member 510 are connected in series, fluid communication is again established from the luer fitting 516 to the connector member 510 via the central bore 520 of the luer fitting 516, the central bore 504 of the central handle member 502 and the central bore 514 of the connector member 510. In this configuration, suction is again established through the handle assembly 500 in the proximal direction of arrow S by connection to a continuous suction source such as an operating room such system (not shown). Bone dust as represented by arrow B at bone dust removal structure 150 is collected by the bone dust collection device 100 or 200 by the establishment of the suction condition S. The fluid extraction flow interruption member 130 (see FIG. 3) again is now in an open position to permit the passage of fluid through the handle assembly 500 while, in contrast to the sealing provided by plug member 164, the fluid flow diversion interruption device 400 is in a closed position. Bone dust is collected in the bone dust collection device 110 or 210.

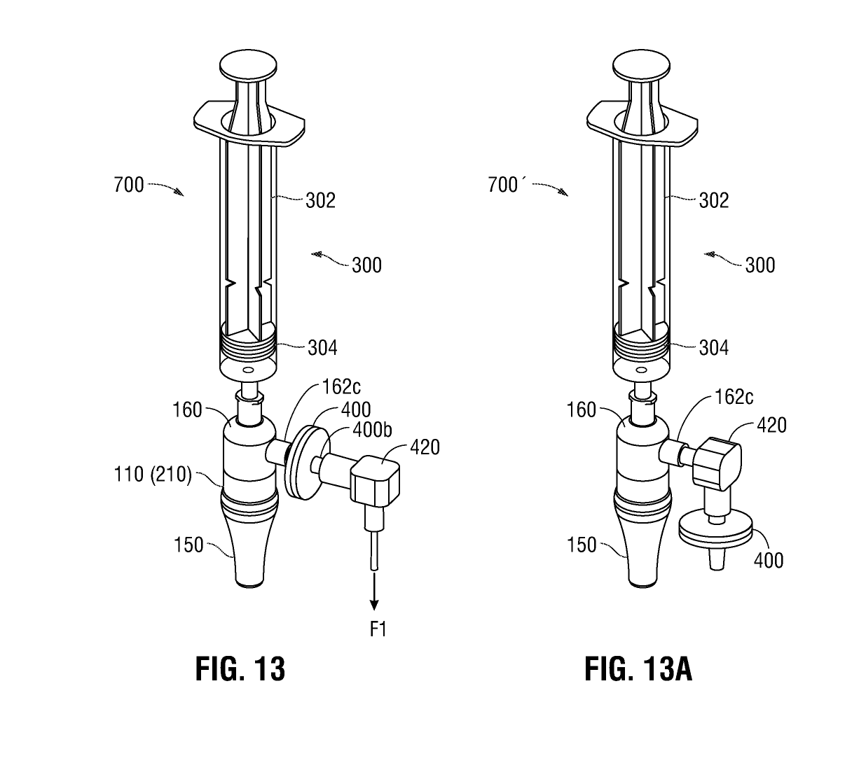

FIG. 13 is a partially transparent view of one embodiment of a self-contained bone dust collection device according to the present disclosure. More particularly, referring to FIG. 4 above, self-contained bone dust collection device 700 includes the suction device 300 which may be a syringe 302 including a plunger 304 in fluid communication with the bone dust collection structure 110 (see FIG. 3) or bone dust collection structure 210. However, as described above with respect to FIG. 5, in lieu of fluid conduit 410, self-contained bone collection device 700 includes flow diversion member 420 that is in fluid communication with the fluid flow diversion interruption device 400 on the downstream side 400b.

Again, flow diversion member 420 may be a right angle or 90 degree luer valve and is further described below. When the plunger 304 is advanced distally following retraction and accumulation of fluid within the syringe 302, fluid flow diversion interruption device 400 shifts to the open position (while fluid extraction flow interruption member 130 shifts to the closed position) to divert fluid through flow diversion member 420. Flow diversion member 420 may be utilized to divert fluid back to a surgical site by directing the fluid vertically downward as shown by arrow μl.

FIG. 13A is a partially transparent view of another embodiment of the self-contained bone collection device of FIG. 13. More particularly, bone dust collection device 700′ differs from bone dust collection device 700 in that fluid flow diversion interruption device 400 is now downstream of flow diversion member 420.

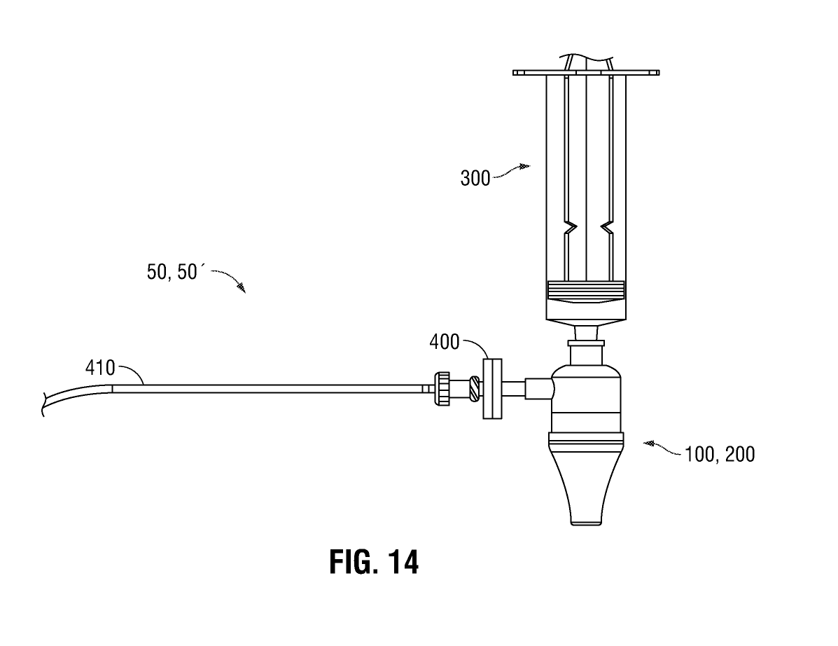

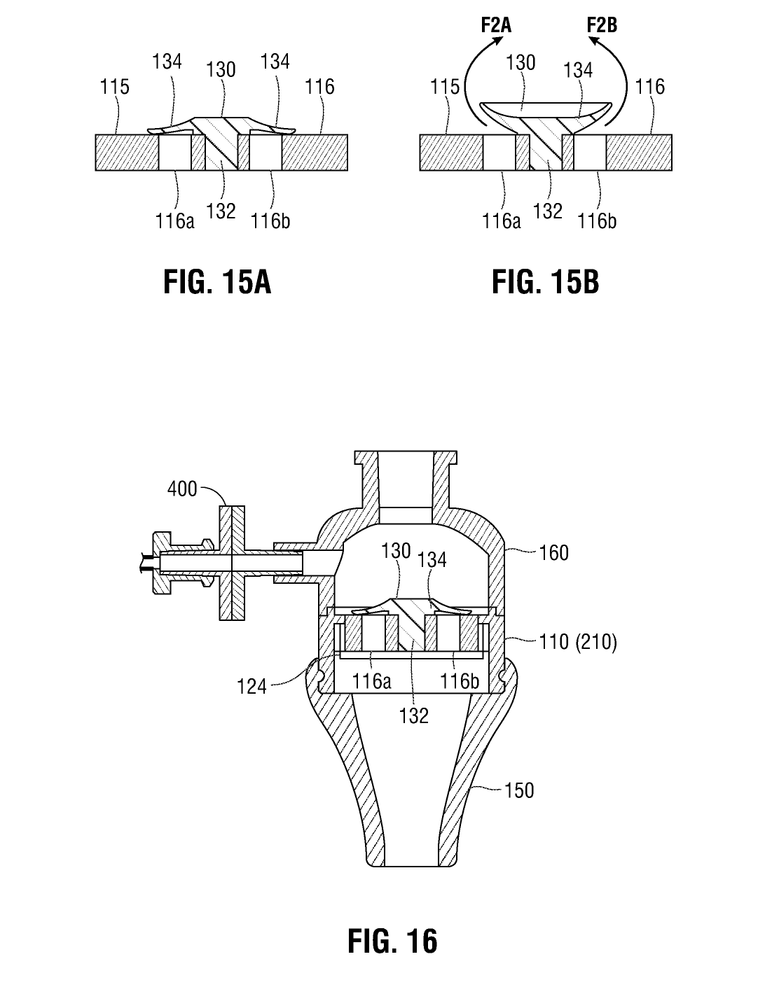

FIG. 14 is a zoomed out partial profile cross-sectional view of the bone dust collection systems 50 or 50′ that includes fluid conduit 410 of FIG. 1-3. FIG. 15A is a detailed view of extraction flow interruption member 130 illustrated in the form of umbrella valve 130 that is positioned on upper surface 115 of the housing in a closed position. The extraction flow interruption member 130 again includes diaphragm 134 that is formed with the shaft member 132. The diaphragm 134 is again made from an elastomeric and flexible material and which overlays the fluid flow apertures 116a and 116b on the proximal side or upper surface 115 of transverse plate member 116. FIG. 15B is a detailed view of the umbrella valve positioned on the upper surface 115 of the housing in an open position to enable flow of fluid past the umbrella valve. Again, the change of position of the extraction flow interruption member 130 occurs via the suction pressure induced on the flow interruption member, and more particularly on the diaphragm 134, such that the diaphragm 134 flips away from the transverse plate member 116 to expose the fluid flow apertures 116a and 116b, thereby enabling flow of fluid through the apertures 116a and 116b, as indicated by arrows F2A and F2B, respectively. Those skilled in the art will recognize that the actual flexing and flipping of the diaphragm 134 to establish the open position is a very small rise away from the transverse plate member 116. That is, the diaphragm 134 must flex upwardly sufficiently to enable passage of the fluid, such as blood and/or saline solution, with any particulates including bone dust or other anatomical specimens contained therein that have passed through the filtration member 124 (see FIG. 3) while the anatomical specimen is being collected. The umbrella valve may be made from thermoplastic elastomers such as, but not limited to, polyvinyl chloride or urethane or the like.

FIG. 16 is a cross-sectional view of the bone dust collection device 100 or 200 when the umbrella valve 130 is in the closed position as described above with respect to FIG. 15A.

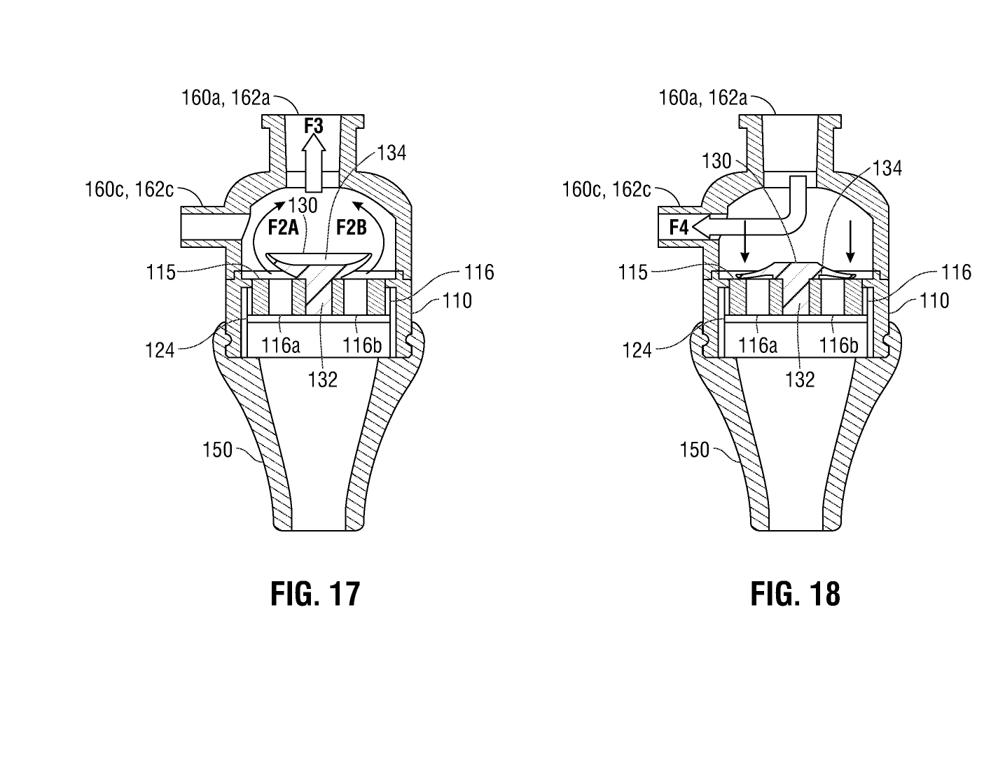

FIG. 17 is a cross-sectional view of the bone dust collection device 100 or 200 when the umbrella valve 130 is in the open position for permitting passage of fluid past the umbrella valve 130 as shown by arrows F2A and F2B and then through suction device interface aperture 162a at proximal portion 160a as shown by arrow F3. In this configuration, the suction device 300 which may be in the form of syringe 302 as described above draws suction on the interface aperture 162a to enable the fluid to be drawn into the syringe. During this operation, the plunger 304 is retracted proximally through the syringe 302.

FIG. 18 is a cross-sectional view of the bone dust collection device 100 or 200 when the umbrella valve 130 is in the closed position for discharging and diverting fluid which has been collected by syringe 302 in the direction of arrow F4 through fluid diversion aperture 162c at branch connection 160c. During this operation, the plunger 304 is advanced distally through the syringe 302.

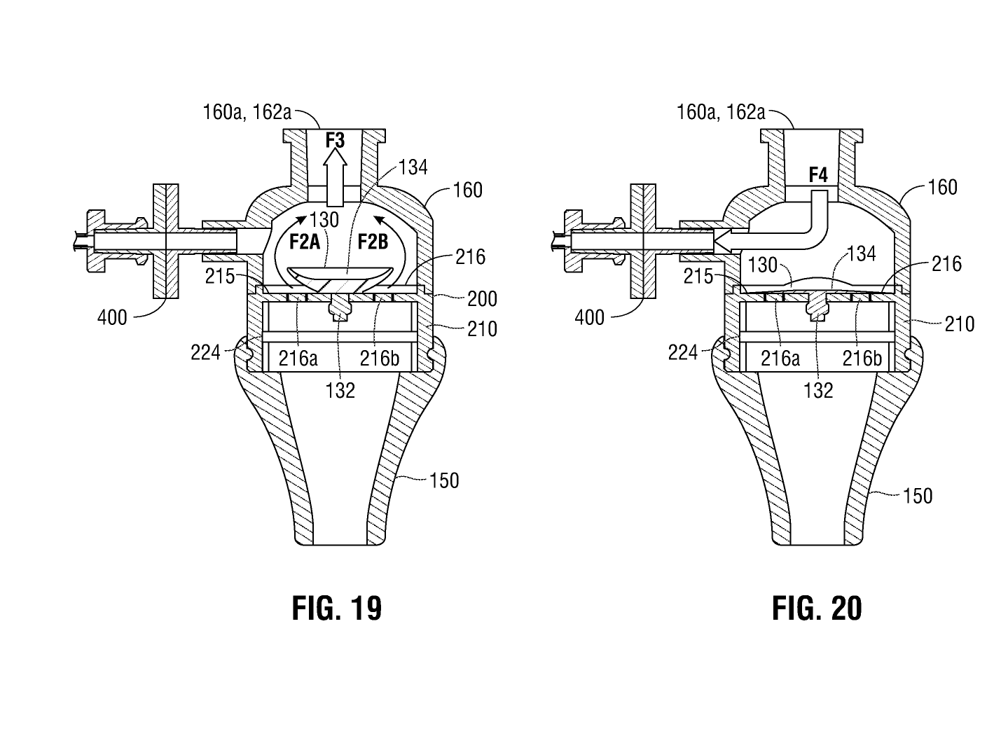

FIG. 19 is a cross-sectional view of another embodiment of the bone dust collection device 200 having the alternate filtration member 224 shown in FIG. 4. During this operation, the fluid flow diversion interruption device 400 is in the closed position and the umbrella valve 130 is in the open position. for permitting passage of fluid past the umbrella valve 130 as shown by arrows F2A and F2B and then through suction device interface aperture 162a at proximal portion 160a as shown by arrow F3.

FIG. 20 is cross-sectional view of the bone dust collection device 200 of FIG. 19 wherein the umbrella valve 130 is in the closed position and fluid is diverted through fluid diversion aperture 162c and the fluid flow diversion interruption device 400 in the direction of arrow F4.

Although the flow interruption member 130 has been illustrated as an umbrella valve, other flow interruption members may be employed such as a miter valve or duck bill valve, a check or flapper valve, or a disk valve or the like. In such cases, the tee-shaped port member or cap 160 and/or the filtration assembly 224 may be reconfigured if or as necessary to accommodate the design, operation and configuration of the particular flow interruption member being employed.

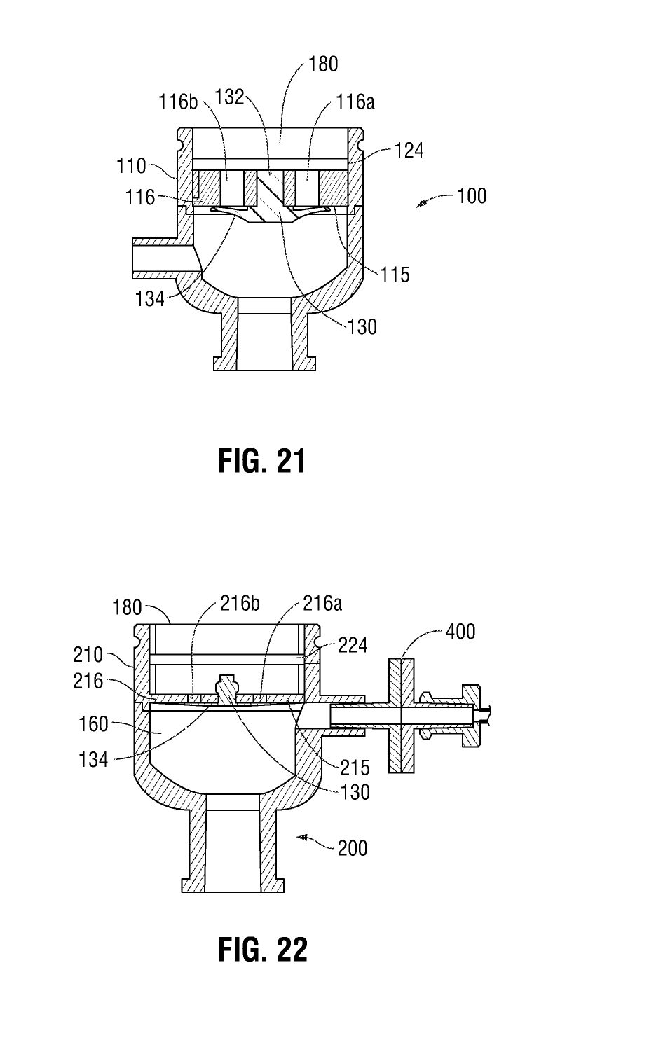

FIG. 21 is a cross-sectional view of the bone dust collection device 100 having the filtration member 124 of FIG. 5 in an inverted position to allow removal of collected bone dust 180. The collected bone dust 180 may be removed manually with a flat instrument or spatula or by a power-assisted instrument including a suction source.

FIG. 22 is a cross-sectional view of the bone dust collection device 200 of FIG. 19 in an inverted position to allow removal of collected bone dust 180. Similarly, the collected bone dust 180 may be removed manually with a flat instrument or spatula or by a power-assisted instrument including a suction source.

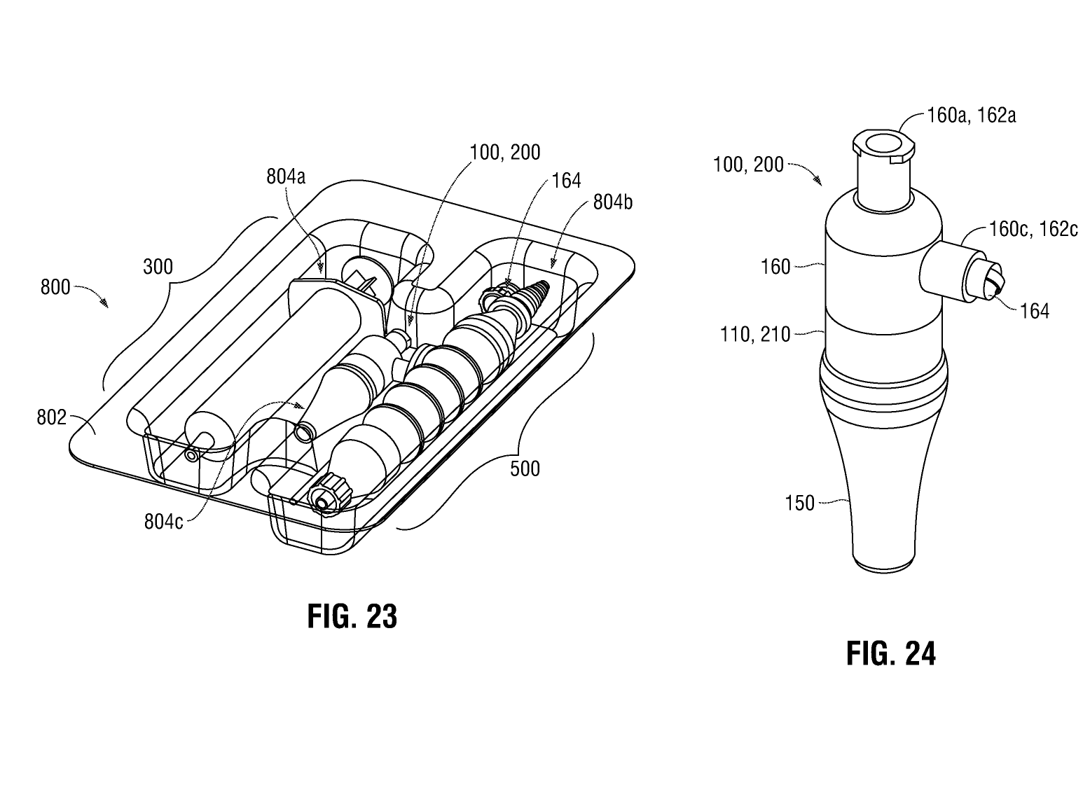

FIG. 23 is a perspective transparent view of a kit package 800 that houses the bone dust collection devices 100 or 200 and associated components according to embodiments of the present disclosure.

More particularly, kit 800 includes a support tray 802 that defines a first depression 804a and an adjacent second depression 804b which are interconnected via a central depression 804c. As illustrated, suction device 300 is received in first depression 804a, handle assembly 500 is received in second depression 804b together with plug member 164 while bone dust collection device 100 or 200 may be received in central depression 804c. When provided with a covering (not shown), the kit package and contents may be provided to the user in a sterile condition via means known in the art.

Alternatively, the contents may also be provided in a sterile condition in a taped banded bag such as, for example, a Sklar banded bag product no. 96-5582 or equivalent as necessary due to the size of the contents (not shown—by Sklar Instruments, West Chester, Pa., USA) or Tyvek® (DuPont de Nemours, Inc.) pouch or equivalent as known in the art.

FIG. 24 is a perspective view of the bone dust collection device 100 of FIG. 3 having plug member 164 to seal off flow through fluid diversion aperture 162c at branch connection 160c during connection of the bone dust collection device to a continuous vacuum system.

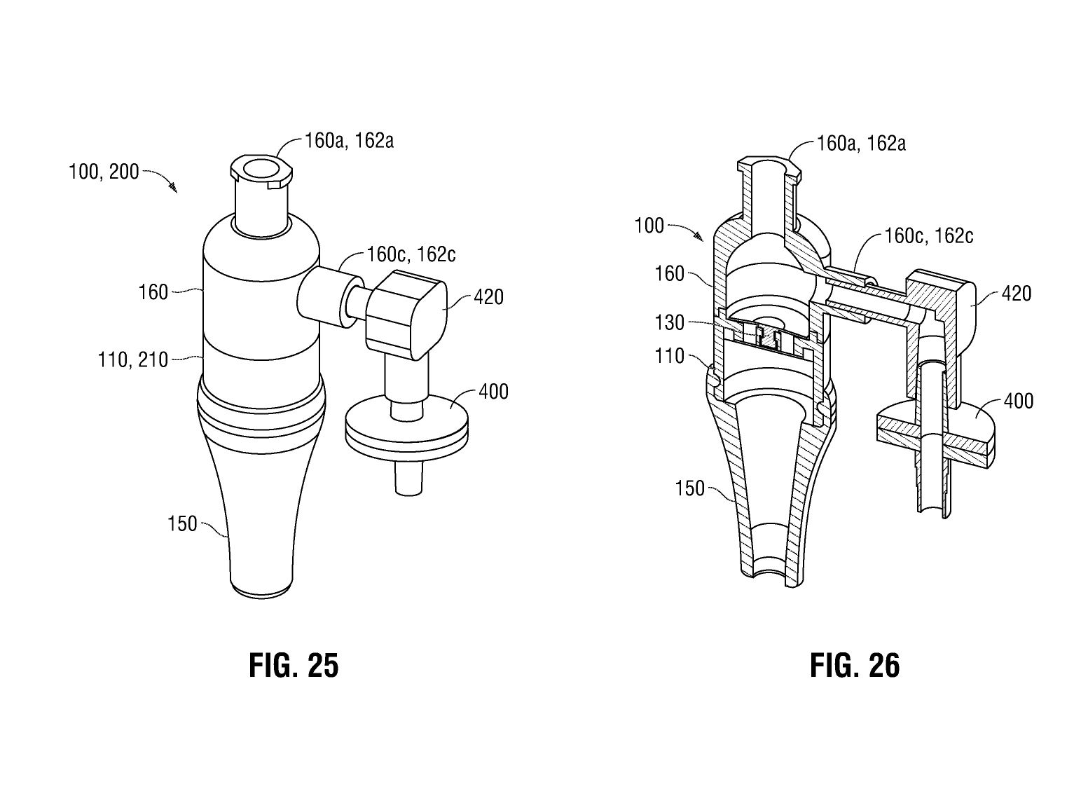

FIG. 25 is a perspective view of the bone dust collection device 100 or 200 of FIG. 24 wherein the plug member 164 is replaced by flow diversion member 420 in the form of a 90 degree elbow and fluid flow diversion interruption device 400 to enable diversion of fluid in a vertically downward direction back to a surgical site.

FIG. 26 is a cross-sectional view of the bone dust collection device 100 of FIG. 25 with fluid flow diversion member 420 in the form of a 90 degree elbow and fluid flow diversion interruption device 400 to enable diversion of fluid in a vertically downward direction back to a surgical site.

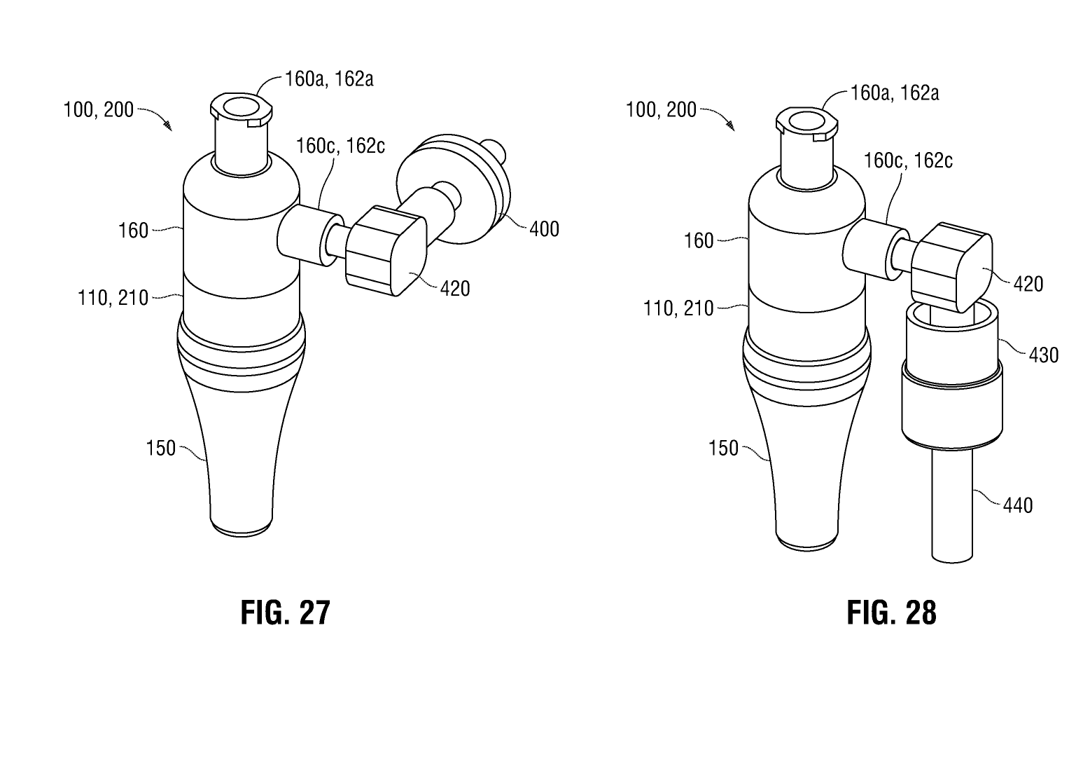

FIG. 27 is a perspective view of the bone dust collection device 100 or 200 of FIGS. 25 and 26 wherein the fluid flow is directed horizontally.

FIG. 28 is a perspective view of the bone dust collection device 100 or 200 of FIG. 25-27 wherein the fluid flow diversion interruption device 400 is replaced by a fluid port connection fitting member 430 and a tubular member 440 to enable diversion in a vertically downward direction of fluid back to a surgical site.

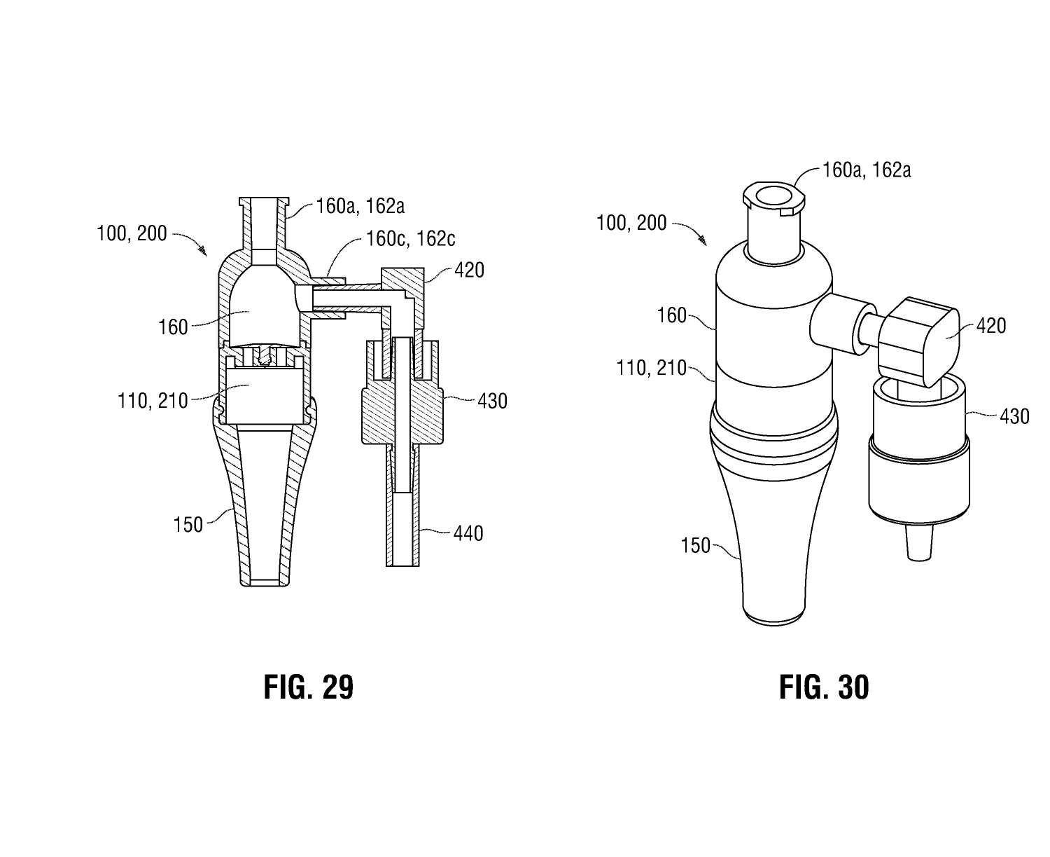

FIG. 29 is a cross-sectional elevation view of the bone dust collection device 100 or 200 of FIG. 28 wherein fluid port connection fitting member 430 and tubular member 440 enable diversion of fluid in a vertically downward direction.

FIG. 30 is a perspective view of the bone dust collection device 100 or 200 of FIG. 29 wherein tubular member 440 of the fluid port connection fitting member 430 has been removed.

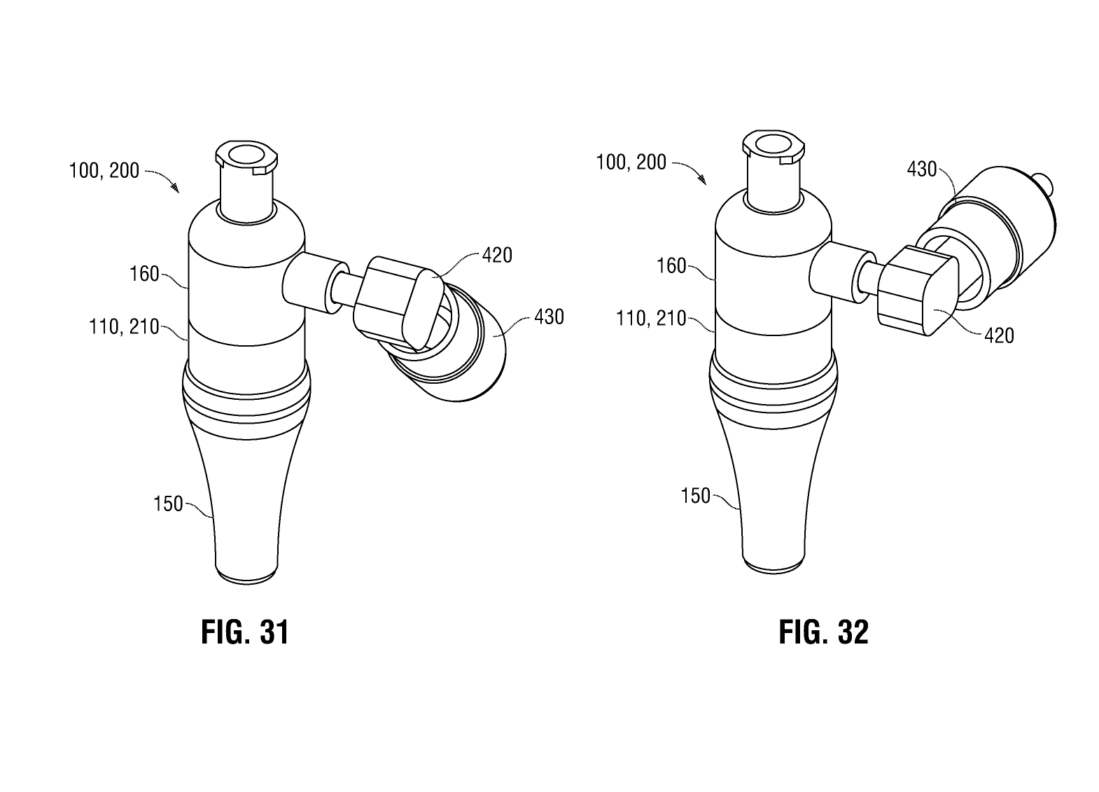

FIG. 31 is a perspective view of the bone dust collection device 100 or 200 of FIG. 30 wherein the flow diversion member 430 is oriented at an angle of 45 degrees below the horizontal to enable diversion of fluid in an alternative direction with respect to a surgical site.

FIG. 32 is a perspective view of the bone dust collection device 100 or 200 of FIG. 28 wherein the flow diversion member 430 is oriented horizontally to enable diversion of fluid in yet another alternative direction with respect to a surgical site.

From the foregoing description, it can be appreciated that the harvested or collected bone may be used during:

(1) Fusion construct—spine or long bone—to promote fusion healing.

(2) Bone fusion wherein placement of bone dust into a fusion device such as a cage implant may be utilized to promote bone fusion.

(3) Replacement of bone after craniotomy or any skull base procedure where bone is removed.

(4) Dental reconstruction/fusion procedure.

Between the bone dust collection structure and the syringe, the one-way valve or check valve allows for continued suction and expulsion of air without disconnecting the syringe from the bone dust collection device.

The size of the central aperture and volume 120 (see FIG. 3) and 220 (see FIG. 4) for receiving and collecting bone dust can be altered depending on use of the device. The size of the bone collection devices 110 and 210 (see FIGS. 3 and 4, respectively) and respective central apertures and volumes 120 and 220 can be made smaller for cervical surgery or made larger for lumbar surgery or long bone repair.

Embodiments of the bone dust collection device may include a mechanism to include or mix bone marrow aspirate or also other biological cells.

Since bone marrow is flexible tissue, embodiments of the bone dust collection device may be used to filter bone marrow alone as a separate device and without the need for drilling. In such embodiments, the filter design parameters may be adjusted accordingly to suit collection of bone marrow or other biological cells.

The bone dust collecting structures 110 or 210 may be centrifuged to spin down bone products and separate blood products.

The harvesting or bone dust collection system or self-contained device generally is provided via a sterile cartridge, adaptable to a standard luer fitting. The harvesting system may be provided sterile by the medical device manufacturer.

The filter assembly allows blood and fluids to pass, but generally retains bone particles between about 75 to about 300 microns. Filter mesh size may be optimized, such as by empirical testing of various filter sizes and types, to result in minimal fluid flow restriction and clogging during the procedure and to result in maximal bone fragment collection. Consideration must also be given to optimization and alternate particle size ranges when collecting other anatomical specimens such as bone marrow or other biological cells.

The bone dust collection volume generally is about 0.5 cc (cubic centimeters) minimum.

The device may be designed to allow a clinician to cycle the syringe multiple times while harvesting bone dust, while dispensing blood/saline out of the side port.

The bone dust harvesting system allows easy removal of the bone dust from the device. The device allows cleaning of the bone dust using a sterile saline flush. The harvesting system allows easy consolidation and removal of the bone dust. Removal methods include utilizing a scoop (spatula, spoon or similar). Prior to removal of the bone dust, aspiration of the bone dust via air flow may assist in drying the bone dust to result in a more cohesive bone plug when removing the bone dust.

The device is generally provided sterile in a double Tyvek® pouch in order to comply with sterile transfer protocol in the sterile OR field.

Sterilization methods may include exposure to Gamma radiation.

The bone dust collection device may be reused on the same patient during the same surgical procedure. The device generally should not be re-sterilized. It is intended for use in the collection of bone dust created during a surgical procedure when using any high-speed drill that creates bone dust (cutting burrs, diamond burrs, etc.).

The device generally does not contain latex.

Bone dust collected by the surgeon is used for the procedure at his or her discretion. As indicated above, bone dust can be packed into an interbody cage device to augment a fusion construct. In conjunction with FIG. 11 or FIG. 12 and the foregoing disclosure.

TABLE 1 below indicates detailed method steps for “Using the Bone Dust Collection System with Operating Room Suction.”

TABLE 1
USING THE BONE DUST COLLECTION SYSTEM WITH
OPERATING ROOM SUCTION

| Step No. | Description |
|---|---|
| 1. | Open the single-pouch in a sterile fashion onto the |
| | operating room surgical field. |
| 2. | Insert side plug into valve body of the device. |
| 3. | Attach handle assembly to the luer lock of the device. |
| 4. | Attach operating room suction tubing to universal |
| | connector on handle assembly. |
| 5. | Using a high-speed drill, create bone dust based on the |
| | procedure. |
| 6. | Irrigate surgical site with saline to suspend the bone dust. |
| 7. | Place the tip of the collection device into the saline/bone |
| | dust mixture and aspirate using suction system. |
| 8. | Collect bone dust in the chamber and saline with blood |
| | products in the suction canister. |
| 9. | Upon completely filling the collection chamber with bone |
| | dust, remove the tip and collect the bone dust with a flat |
| | instrument or spatula. |
| 10. | Pack the collected bone dust into the region of interest in |
| | the subject - interbody cage, bone defect, etc. |
| 11. | If necessary, replace tip and collect additional bone dust. |
| 12. | If necessary, repeat usage of the bone dust collection |
| | system until enough bone for the procedure has been |
| | collected. |

In conjunction with FIG. 13 or FIG. 13A, and the foregoing disclosure, TABLE 2 below indicates detailed method steps for “Using the Self-Contained Bone Dust Collection Device with Syringe Suction.”

TABLE 2
USING THE SELF-CONTAINED DEVICE
WITH SYRINGE SUCTION

| Step No. | Description |
|---|---|
| 1. | Open the single-pouch in a sterile fashion onto the |
| | operating room surgical field. |
| 2. | Attach a 30 cc syringe to the luer lock of the device. |
| 3. | Attach the right angle check valve assembly to the valve |
| | body. |
| 4. | Using a high-speed drill, create bone dust based on the |
| | procedure. |
| 5. | Suspend the bone dust using a small amount of saline |
| | irrigation. |
| 6. | Place the tip of the collection device into the saline/bone |
| | dust mixture and aspirate by pulling back the syringe |
| | piston. Bone dust is collected in the chamber while saline |
| | and blood products are collected within the syringe. |
| 7. | Recycle excess saline and blood collected within the |
| | syringe by pushing in the syringe piston allowing the |
| | excess fluid to be expelled through the side port back into |
| | the surgical wound. |
| 8. | When the collection chamber is filled with bone dust, |
| | remove the tip and collect bone dust with a flat instrument |
| | or spatula. |
| 9. | Pack the collected bone dust into the region of interest in |
| | the subject - interbody cage, bone defect, etc. |
| 10. | If necessary, replace tip and collect additional bone dust. |
| 11. | If necessary, repeat usage of the bone dust collection |
| | system until enough bone for the procedure has been |
| | collected. |

In view of the foregoing, the embodiments of the present disclosure relate to a general method of collecting an anatomical specimen that includes the following steps.

With reference particularly to FIGS. 11, 12, 13 and 13A, the method includes generating an anatomical specimen, e.g., bone dust 180 (see FIG. 21) in particulate form at a surgical site of a subject (not shown).

The method includes irrigating the surgical site to suspend the particulate in a particulate fluid mixture (not shown). Upon irrigating the surgical site, the method includes disposing a tip of a collection device, e.g., the collection device 100 in FIG. 1, in the particulate fluid mixture (e.g., represented in FIG. 15B by arrows F2A and F2B internally within the collection device 100 in FIG. 1); aspirating the collection device, e.g., collection device 100, to filter and separate the particulate from the particulate fluid mixture therein accumulating the particulate, e.g., bone dust 180 in FIG. 17, in the collection device, e.g., collection device 100; and collecting the particulate, e.g., bone dust 180, accumulated in the collection device 100.

The method may further include packing the collected particulate, e.g., bone dust 180, into a region of interest in the subject (not shown).

The step of collecting the particulate accumulated in the collection device may be preceded by disassembling the collection device. The method may further include reassembling the collection device and repeating the foregoing steps of generating additional anatomical specimen in particulate form at a surgical site of the subject; irrigating the surgical site to suspend the particulate in a particulate fluid mixture; disposing the tip of the collection device in the particulate fluid mixture; aspirating the collection device to filter and separate the particulate from the particulate fluid mixture therein accumulating the particulate in the collection device; and collecting the particulate accumulated in the collection device.

The method may then include again packing the additionally collected particulate, e.g., bone dust 180, into a region of interest in the subject.

As shown in FIGS. 11 and 12, the aspirating of the collection device 100 may include connecting the collection device 100 to a vacuum source, that establishes suction through the handle assembly 500 in the proximal direction of arrow S in an operating room (not shown) wherein the vacuum source is in fluid communication with the fluid in the particulate fluid mixture, shown by arrow B, to filter and separate the particulate, bone dust 180 from the particulate fluid mixture, shown by arrow B.

Alternatively, as shown in FIGS. 13 and 13A, the aspirating of the collection device 100 may include actuating a syringe, e.g., syringe 300, in fluid communication with the collection device 100 and in fluid communication with the particulate fluid mixture, shown by arrow B, wherein the actuating of the syringe effects filtering and separating the particulate, e.g., bone dust 180, from the particulate fluid mixture.

In conjunction with the foregoing disclosure and FIGS. 1-32, TABLE 3 indicates “Consideration for Using the Bone Dust Collection System or Device”.

TABLE 3
CONSIDERATIONS FOR USING THE BONE DUST
COLLECTION SYSTEM OR DEVICE

| Considera- | |
|---|---|
| tion No. | Description |
| 1. | The device functions better with saline and bone dust |
| | mixture while minimizing the amount of blood. |
| 2. | If there is too much blood in the field, the tip or filter may |
| | become clogged making it difficult to aspirate bone dust. |
| | The surgeon should irrigate the field, stop bleeding as |
| | much as possible, and try the procedure for bone dust |
| | collection at this point with a dry field. |
| 3. | The tip may detach with excessive force while pushing into |
| | the wound to collect bone dust. Simply re-attach. |
| 4. | Occasionally, the tip can become clogged with minimal |
| | saline. Irrigate further and simply put gentle pressure on |
| | the tip to dislodge stuck bone dust. |
| 5. | The device is not a fusion device; it is intended to assist |
| | the surgeon in collecting bone dust. |
| 6. | It is not expected that the device promotes better fusion |
| | rates. |

The detailed description of exemplary embodiments herein makes reference to the accompanying drawings, which show the exemplary embodiments by way of illustration and their best mode. While these exemplary embodiments are described in sufficient detail to enable those skilled in the art to practice the disclosure, it should be understood that other embodiments may be realized and that logical and mechanical changes may be made without departing from the spirit and scope of the disclosure. Thus, the detailed description herein is presented for purposes of illustration only and not of limitation. For example, the steps recited in any of the method or process descriptions may be executed in any order and are not limited to the order presented. Moreover, any of the functions or steps may be outsourced to or performed by one or more third parties. Furthermore, any reference to singular includes plural embodiments, and any reference to more than one component may include a singular embodiment.

1. A bone dust collection device comprising:
a bone dust collection structure defining a longitudinal axis and having a proximal end and a distal end,
the bone dust collection structure having a transverse plate member connected at or in the vicinity of the proximal end to a cylindrical wall defining an aperture at the distal end for receiving and collecting bone dust, the aperture extending proximally and including a filtration member disposed therein, the filtration member configured and disposed wherein the filtration member causes at least partial separation of fluid from the bone dust via the fluid passing proximally through the filtration member, the bone dust collection structure configured to enable removal of bone dust collected therein.

2. The bone dust collection device according to claim 1, wherein at least one aperture is defined in the transverse plate member that enables the fluid at least partially separated from the bone dust by the filtration member to pass therethrough.

3. The bone dust collection device according to claim 2, further comprising a flow interruption member configured and disposed with respect to the bone dust collection structure wherein flow of fluid through the at least one aperture of the bone dust collection structure occurs by suction pressure induced on the flow interruption member causing the flow interruption member to change position to enable flow through the at least one aperture defined in the transverse plate member.

4. The bone dust collection device according to claim 3, wherein at least another aperture is defined in the transverse plate member wherein the flow interruption member includes a shaft member disposed through the at least another aperture wherein the change of position of the flow interruption member occurs via the suction pressure induced on the flow interruption member to enable flow through the at least one aperture defined in the transverse plate member.

5. (canceled)

6. (canceled)

7. (canceled)

8. (canceled)

9. The bone dust collection device according to claim 3, further comprising another flow interruption device configured and disposed with respect to the flow interruption device and with respect to the bone dust collection structure wherein suction pressure induced on the another flow interruption device causes the another flow interruption device to be in a closed position and causes the flow interruption member to change position to enable flow through the at least one aperture defined in the transverse plate member.

10. The bone dust collection device according to claim 3, further comprising another flow interruption device configured and disposed with respect to the flow interruption device and with respect to the bone dust collection structure wherein the another flow interruption device is a plug member to seal off flow therethrough during connection of the bone dust collection device to a continuous vacuum system and suction pressure induced on the flow interruption device causes the flow interruption member to change position to enable flow through the at least one aperture defined in the transverse plate member.

11. The bone dust collection device according to claim 1, further comprising:
a bone dust removal structure defining an aperture having a proximal end and a distal end, the aperture configured and disposed for facilitating transport of bone dust from the distal end of the bone dust removal structure to the bone dust collection structure.

12. The bone dust collection device according to claim 11, wherein the bone dust removal structure includes a nozzle that defines the aperture of the bone dust removal structure, the nozzle defining a proximal end and a distal end, wherein the nozzle facilitates transport of the bone dust to the bone dust collection structure.

13. The bone dust collection device according to claim 12, wherein the nozzle is removably attached to the bone dust collection structure, wherein removal of the nozzle from the bone dust collection structure enables removal of bone dust collected within the bone dust collection structure.

14. The bone dust collection device according to claim 3, further comprising a guide member configured and disposed with respect to the suction source and the bone dust collection structure for enabling a user to manually control the location of the bone dust removal structure during a surgical procedure.

15. The bone dust collection device according to claim 3, wherein the flow interruption member is an umbrella valve.

16. The bone dust collection device according to claim 9, wherein the another flow interruption member is a check valve.

17. A bone dust collection device comprising:
a bone dust collection structure defining a proximal end and a distal end, the bone dust collection structure including a filtration member at the proximal end and defining an aperture at the distal end for receiving and collecting bone dust, the filtration member causing at least partial separation of fluid from the bone dust via the fluid passing proximally through the filtration member; and
a bone dust removal structure defining an aperture having a proximal end and a distal end, the aperture configured and disposed for facilitating transport of bone dust from the distal end of the bone dust removal structure to the bone dust collection structure, the bone dust removal structure removably attached to the bone dust collection structure, wherein removal of the bone dust removal structure from the bone dust collection structure enables removal of bone dust collected within the bone dust collection structure.

18. An anatomical collection system comprising:
an anatomical specimen collection structure defining a proximal end and a distal end, the anatomical specimen collection structure including a filtration member at the proximal end and defining an aperture at the distal end for receiving and collecting an anatomical specimen, the filtration member causing at least partial separation of fluid from the anatomical specimen via the fluid passing proximally through the filtration member; and
an anatomical specimen removal structure defining an aperture having a proximal end and a distal end, the aperture configured and disposed for facilitating transport of the anatomical specimen from the distal end of the anatomical specimen removal structure to the anatomical specimen collection structure, the anatomical specimen removal structure removably attached to the anatomical specimen collection structure wherein detachment of the anatomical specimen removal structure from the anatomical specimen collection structure enables removal of the anatomical specimen collected within the anatomical specimen collection structure.

19. The anatomical collection system according to claim 18, wherein the anatomical specimen is bone dust.

20. A method of collecting an anatomical specimen comprising:
generating an anatomical specimen in particulate form at a surgical site of a subject;
irrigating the surgical site to suspend the particulate in a particulate fluid mixture;
disposing a tip of a collection device in the particulate fluid mixture;
aspirating the collection device to filter and separate the particulate from the particulate fluid mixture therein accumulating the particulate in the collection device; and
collecting the particulate accumulated in the collection device.

21. The method according to claim 20, further comprising:
packing the collected particulate into a region of interest in the subject.

22. The method according to claim 20, wherein the collecting the particulate accumulated in the collection device is preceded by disassembling the collection device, the method further comprising:
reassembling the collection device;
generating additional anatomical specimen in particulate form at a surgical site of the subject;
irrigating the surgical site to suspend the particulate in a particulate fluid mixture;
disposing the tip of the collection device in the particulate fluid mixture;
aspirating the collection device to filter and separate the particulate from the particulate fluid mixture therein accumulating the particulate in the collection device; and
collecting the particulate accumulated in the collection device.

23. The method according to claim 22, further comprising:
packing the additionally collected particulate into a region of interest in the subject.

24. The method according to claim 20, wherein the aspirating of the collection device includes connecting the collection device to a vacuum source wherein the vacuum source is in fluid communication with the fluid in the particulate fluid mixture to filter and separate the particulate from the particulate fluid mixture.

25. The method according to claim 24, wherein the connecting the collection device to a vacuum source includes connecting the collection device to a vacuum source in an operating room.

26. (canceled)
